(19) United States
(12) Patent Application Publication
(10) Pub. No.: US 2024/0307560 A1
(43) Pub. Date: Sep. 19, 2024

(54) HEPTAMETHINE CARBOCYANINE DYE-DOTA CONJUGATES COMPLEXED WITH LUTETIUM-177, YTTRIUM-90, OR GALLIUM-68, AND THEIR USES FOR IMAGE-GUIDED RADIOTHERAPY

(71) Applicant: Da Zen Theranostics, Inc., Beverly Hills, CA (US)

(72) Inventors: Leland W.K. Chung, Beverly Hills, CA (US); Ruoxiang Wang, Los Angeles, CA (US); Yi Zhang, Los Angeles, CA (US)

(21) Appl. No.: 18/270,463

(22) PCT Filed: Dec. 30, 2021
(86) PCT No.: PCT/US2021/065651

§ 371 Date: Jun. 29, 2023

Related U.S. Application Data
(60) Provisional application No. 63/132,724, filed on Dec. 31, 2020.

(51) Int. Cl. A61K 51/04

(52) U.S. Cl. CPC A61K 51/0446; A61K 51/0406; A61K 51/0497

(57) ABSTRACT

Embodiments of the present invention generally relate to conjugates of heptamethine carbocyanine dye (HMCD)-chelator radiometal complexes, radiopharmaceutical formulations comprising such complexes and their use, in particular for internal radiotherapy and/or imaging of cancer. In particular, in embodiments, provided are DZ-1-Lys-DOTA conjugates complexed with Lutetium-177, Yttrium-90 or Gallium-68, or combinations thereof. Some embodiments provide improved radiotherapy with complexes of Lutetium-177 or Yttrium-90. Further embodiments provide improved radioimaging with Gallium-68 complexes, and their use. Yet further embodiments provide improved image-guided therapy by using matched theranostic pairs of such conjugates, wherein for therapy purposes the radiometal is selected from one or more of Lutetium-177, Yttrium-90, each of which for imaging purposes may be paired with Gallium-68, for improved image-guided radiotherapy.

### FIELD OF THE INVENTION

Embodiments of the present invention generally relate to conjugates of the near infrared (NIR) heptamethine carbocyanine dye (HMCD) with chelator-radiometal complexes, radiopharmaceutical formulations comprising such complexes and their use, e.g. in internal radiotherapy and/or imaging of cancer. In particular, in embodiments, provided are DZ-1-Lys-DOTA conjugates complexed with Lutetium-177, Yttrium-90 or Gallium-68, or combinations thereof. Some embodiments provide improved radiotherapy with complexes of Lutetium-177 or Yttrium-90. Further embodiments provide improved radioimaging with Gallium-68 complexes.

Yet further embodiments provide improved image-guided therapy by using matched theranostic pairs of such conjugates, wherein for therapy purposes the radiometal is selected from one or more of Lutetium-177, Yttrium-90, each of which for imaging purposes may be paired with Gallium-68, for improved image-guided radiotherapy. The present invention also relates to methods of forming the complexes, pharmaceutical compositions comprising the complexes, methods of using the complexes or pharmaceutical compositions, methods of imaging and/or radiotherapy of cancer cells, tissues, soft and solid tumors, and/or their metastases, kits for imaging and/or radiotherapy, and the like. Advantages of the embodiments may include improved imaging and/or tumor detection, improved therapy of tumors, cancer, metastases, and pre-cancerous lesions, improved image guided therapy, improved serum stability, and improved penetration of tumors, especially solid tumors, and/or a better matched affinity of theranostic pairs.

### BACKGROUND

A limited number of diagnostic or therapeutic radiopharmaceuticals is available for imaging or treating cancer or tumors. Imaging takes advantage of the very low concentrations needed for detection, e.g. with Positron Emission Tomography (PET) detecting positron-emitters. Internal radiotherapy is a treatment in which a source of radiation is put inside the subject's body for cancer therapy. Internal radiation therapy may be systemic, i.e. the treatment travels in the blood to tissues throughout the body to kill cancer cells through the radiation that the cells in proximity to the radiometal are exposed to. Radiometals can be delivered by chelating or complexing them with a large variety of chelator compounds and conjugating them to a targeting ligand.

Various targeting ligand, chelator and radiometal combinations have been attempted for use in imaging, radiotherapy, or both, including matched pairs of the same targeting ligand for a theranostic approach. However, affinities of the targeting ligand for its receptor(s) have been found to be strongly dependent both on the chelator and radiometal used, thus their efficacy is unpredictable. Similar unpredictability applies to stability of the radiometal complexes, as retaining the radiometal may be influenced by the targeting ligand, chelator, or the combination thereof, and in turn their combination with a desired radiometal. A particular problem with using matched theranostic pairs of the same pharmacophore (different radiometals but same carrier) is that the reporter (imaging) and the therapeutic should exhibit very similar pharmacokinetics to be suitable for matched-pair use, which may not be the case as individual metal-chelate conjugates can have a distinct influence on affinity and/or stability.

Radionuclide complexes currently used for such targeted radiotherapy are mostly diagnostic, with a limited number of therapeutic applications. The latter include various antibodies, proteins, and some smaller compounds, in particular peptides that are able to target certain types of cancer.

Relative to the use in radiotherapy, radionuclides and radiometals are more often used for tumor imaging, or adding imaging functionality to targeted cancer drugs. Certain dye conjugates are known for targeted cancer drug therapy and imaging from WO 2016106324 and U.S. Pat. No. 10,307,489; these include two particular double-conjugated dye-drug-chelator conjugates wherein the dye is conjugated to a) anti-cancer drug gemcitabine as well as b) an imaging moiety, wherein the imaging moiety is ⁶⁴Cu-DOTA or Gd³⁺-DOTA radiometal. The chelated/complexed radiometals are employed to provide imaging functionality to the cancer drug conjugates.

Similarly, a lysine-linked heptamethine carbocyanine dye conjugate with DOTA radiochemical complexed with Cu-64 (DZ-1-Lys-DOTA-Cu-64) was described for imaging of cancer by Xiao et al., Nuclear Medicine and Biology 40, p. 351-360 (2013).

A problem with known radiometal complexes can be a lack of stability in the body, e.g. in blood and/or serum, which may result in insufficient performance, in particular in imaging performance when trying to detect small secondary tumors or metastases, as well as low efficacy and unnecessarily high radiation exposure.

For example Cu-DOTA complexes are generally unstable for longer periods of time in vivo, however, placing DOTA chelators within a nanostructure increases the stability of the 64Cu-DOTA complex as discussed by Jacques Lux et al., Theranostics, Vol. 5, Issue 3 (2015), who describe nanogels to stabilize 64Cu-DOTA and certain other 64Cu complexes for PET-imaging.

Radiotherapy tends to be even more challenging than mere imaging applications and options for treatment by radiotherapy are thus much more limited. Careful integration is needed for a suitable radionuclide in combination with a suitable chelator and a suitable targeting ligand to provide the desired characteristics including affinity, stability and pharmacokinetics. Particular radiometals may emit one or more type of particles or radiation at particular different percentages, and for suitable targeting careful coordination of carrier with a given radiometal is needed so that the complex is stable, sufficiently effective to destroy the target cancer but arrives at the location without causing collateral radiation damage to the not targeted rest of the body and its organs and tissues.

Some therapeutics can provide both therapy and diagnostic imaging to a degree, though for improved performance of each, theranostic pairs are often employed. A “true” theranostic pair would be an identical matched pair, i.e. have the same carrier for the therapeutic radiometal to destroy the tumor, and the imaging radiometal to allow to diagnostic and/or guided therapy. However, both chelator and radiometal affect important performance characteristics and pharmacokinetics, thus a given carrier may not be able to provide an identically matched pair with suitable characteristics, stability and efficacy for a desired radiometal combination. As an alternative, a non-identical matched pair may be used to adjust characteristics, provided that the binding affinities do not present clinically significant differences.

An example of such a non-identical pair of peptide radiopharmaceuticals used in a theranostic approach for somatostatin receptor (SSTR) positive neuroendocrine tumors (NETs) is Lu-177-DOTA-TATE used with Ga-68-DOTA-TOC, each with a different peptide ligand for tumor cell binding with different affinities that have been reported not to be clinically significant.

A particular problem of radiochemicals is a high and persistent localization of the radioactivity being observed in certain organs, for example, without limitation, the kidneys, which compromises tumor visualization in the kidney region and limits therapeutic potential, especially for kidney tumors and tumors localized in that area. Thus there is an interest in reducing renal radioactivity levels but not those in the target tissue(s). Radionuclide complexes also may encounter resorption by proximal tubules of the kidneys and/or adverse long residence times of radiometabolites in cells, particularly in renal cells, or cells of other organs, which may cause undesirably persistent radioactivity.

Further problems with radiochemicals may include one or more of: lack of suitability for therapy for one or more cancer, insufficient sensitivity, insufficient selectivity for cancer cells, insufficient contrast between tumor and noncancerous tissues over time, insufficient biodistribution, insufficient tumor targeting, insufficient tissue and/or tumor penetration, insufficiently uniform distribution in tumor tissues, insufficient tumor accumulation ratios, insufficient retention in the tumor, slow clearance from the blood after administration, undesirably high radioactive exposure of tissues and organs, low clearance rate from important organs such as kidney, liver, heart etc., high radiation damage of non-targeted tissues or organs, in particular encapsulated tissues such as organs and dense tissues including tumor tissues, in particular calcified tumor tissues, slow establishment of steady state distribution in the body and organs including heart, liver, lungs and kidney, persistence of radioactivity in one or more organs including kidney and/or liver, no stable complex formation with one or more radiometal or difficulties to form stable complexes, low complex formation rates, low labelling efficiency with one or more radiometal, insufficient solubility in aqueous solutions, low stability in aqueous solutions, low stability at physiological pH, low stability in vivo, high rate of dissociation of the radiometal from the complex, in particular in vivo, slow tumor-specific targeting, high organ accumulation (e.g. kidney, spleen, liver, heart), slow clearance (e.g. from blood, liver, kidneys and other organs), and difficult or costly synthesis.

Therefore, there is a continued need in the art for radiopharmaceuticals with new or improved characteristics, or combinations thereof, in particular for radiotherapy that require a more extensive and improved set of characteristics to be suitable for in vivo applications including effectiveness, stability, and sufficient tumor penetration, among others. In particular, there is a need for therapeutic radiopharmaceuticals that provide improved imaging, improved therapy, or both. Specifically, there is a need for better-matched theranostic pairs of equal affinity and sufficient efficacy and stability and to provide an improved image-guided therapy.

These and other features and advantages of the present invention will be explained and will become apparent to one skilled in the art through the summary of the invention that follows.

### SUMMARY OF THE INVENTION

In an embodiment, provided is a DZ-1-Lys-DOTA conjugate radiometal complex, wherein the conjugate comprises a heptamethine carbocyanine dye (HMCD) moiety conjugated with a DOTA moiety via a lysine linker and as shown in FI below:

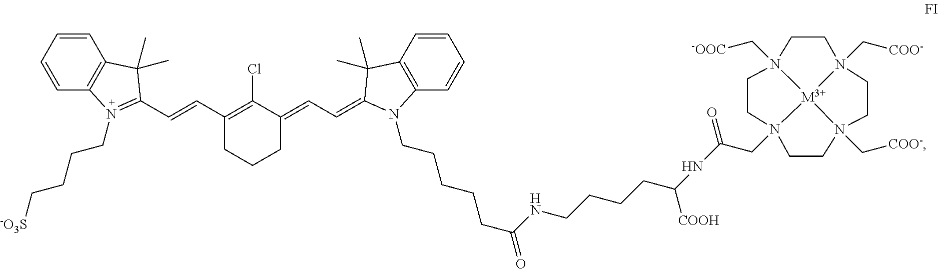

wherein the DOTA moiety is complexed with a radiometal M; and wherein M is selected from one or more radiometal from the group consisting of: Lutetium-177 (Lu-177), Yttrium-90 (Y-90), and Gallium-68 (Ga-68).

In an embodiment, provided are complexes as described herein provided with one or more pharmaceutically acceptable excipient as one or more pharmaceutical formulation.

In an embodiment, provided are complexes as described herein wherein one or more pharmaceutical formulations are adapted for coordinated administration of a first and a second complex to provide an image-guided therapy of cancer, wherein the radiometal of the first complex is Gallium-68, and the radiometal of the second complex is selected from the group consisting of Lutetium-177 and Yttrium-90.

In an embodiment, provided are complexes as described herein provided as a kit with one or more reagents for reconstitution of the first and the second complex in an administrable form.

In an embodiment, provided is a kit for forming one or more complex from a DZ-1-Lys-DOTA conjugate of FII as shown below:

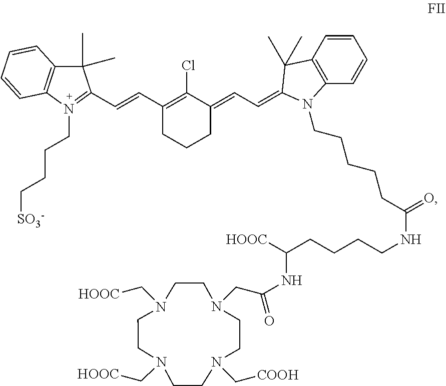

wherein the kit is provided with instructions for mixing and complexing the one or more conjugate in a suitable amount with the one or more radiometal selected from the group consisting of Lu-177, Y-90, and Ga-68 in a suitable amount, optionally with one or more reagent, buffer or excipient, and optionally treating the resulting solution containing the formed complex to provide it in an administrable form.

In an embodiment, provided is a kit for forming a plurality of complexes as described herein for coordinated administration of a first and a second complex, wherein the first complex is Ga-68, and the second complex is selected from the group consisting of Lu-177 and Y-90.

In an embodiment, provided is a method for image-guided therapy of cancer wherein a first and a second DZ-1-Lys-DOTA conjugate radiometal complex of formula I are administered in a coordinated administration schedule to a subject suffering from cancer to provide an image-guided therapy of cancer; wherein the radiometal of the first complex is Gallium-68; wherein the radiometal of the second complex is selected from the group consisting of Lutetium-177 and Yttrium-90; and wherein the first and the second complex has the structure shown in FI below:

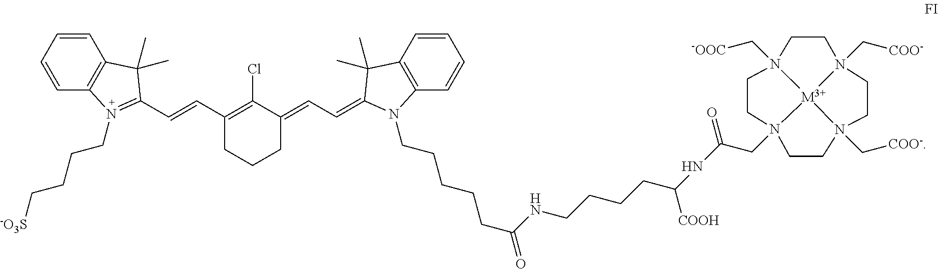

In an embodiment, provided is a wherein imaging of complexes as described herein is performed by Positron Emission Tomography (PET) and optionally by PET and Computer Tomography (CT).

### DESCRIPTION OF THE DRAWINGS

FIG. 1 illustrates the serum stability of DZ-1-Lys-DOTA-6Ga in serum at 37° C. shown by radiochemical purity determined by radio-Thin Layer Chromatography (TLC) over time in minutes. The graph shows a high stability and very slow decline of DZ-1-Lys-DOTA-6Ga.

FIG. 2 shows a graph of the blood clearance of the DZ-1-Lys-DOTA-68Ga probe in Balb/C mice after i.v. injection of 50 μCi of the probe.

FIG. 3 illustrates the serum stability of DZ-1-Lys-DOTA-¹⁷⁷Lu in serum at 37° C. shown by radiochemical purity determined by radio-Thin Layer Chromatography (TLC) over time in minutes. The graph shows a moderate stability and gradually slowing decline of DZ-1-Lys-DOTA-¹⁷⁷Lu with retention of about 30% over the duration of 1400 minutes.

FIG. 4 shows the blood clearance of DZ-1-Lys-DOTA-¹⁷⁷Lu probe in Balb/C mice after i.v. injection of 50 μCi of the probe.

FIG. 5 illustrates the serum stability of DZ-1-Lys-DOTA-⁹⁰Y in serum at 37° C. shown by radiochemical purity determined by radio-Thin Layer Chromatography (TLC) over time in minutes. The graph shows a moderate stability and gradually slowing decline of DZ-1-Lys-DOTA-⁹⁰Y with retention of about 30% over the duration of 1400 minutes.

FIG. 6 shows DZ-1-Lys-DOTA-⁹⁰Y that kills MDA-MB-231 triple-negative breast cancer cells more effectively compared with ⁹⁰Y as evidenced by 48 h data for DZ-1-Lys-DOTA-⁹⁰Y compared to free 90Y data (48 h) at 20 microCi. The same general trend can be seen in other data points with exposure to DZ-1-Lys-DOTA-⁹⁰Y always providing a lower % cell viability.

### DETAILED SPECIFICATION

Embodiments of the present invention generally relate to conjugates of heptamethine carbocyanine dye (HMCD)-chelator radiometal complexes, radiopharmaceutical formulations comprising such complexes and their use, in particular for internal radiotherapy and/or imaging of cancer. In particular, in embodiments, provided are DZ-1-Lys-DOTA conjugates complexed with Lutetium-177, Yttrium-90 or Gallium-68, or combinations thereof. Some embodiments provide improved radiotherapy with complexes of Lutetium-177 or Yttrium-90. Further embodiments provide improved radioimaging with Gallium-68 complexes. Yet further embodiments provide improved image-guided therapy by using matched theranostic pairs of such conjugates, wherein for therapy purposes the radiometal is selected from one or more of Lutetium-177, Yttrium-90, each of which for imaging purposes may be paired with Gallium-68, for improved image-guided radiotherapy. The present invention also relates to methods of forming the complexes, pharmaceutical compositions comprising the complexes, methods of using the complexes or pharmaceutical compositions, methods of imaging and/or radiotherapy of cancer cells, tissues, soft and solid tumors, and/or their metastases, kits for imaging and/or radiotherapy, and the like. Advantages of the embodiments may include improved imaging of cancer, e.g. tumor imaging, improved therapy of cancer, tumors and metastases including pre-cancerous lesions, and, improved image guided therapy, improved serum stability, improved penetration of tumors, especially solid tumors, and/or a matched affinity of theranostic pairs.

The DZ1-Lys-DOTA-radiometal complexes provided in embodiments may be referred to herein as “conjugate complexes”, “dye complexes”, “DOTA complexes”, “dye-DOTA complexes”, “HMCD-DOTA complexes”, “DZ1-DOTA complexes”, “radiometal complexes”, or simply “complexes”.

Without wishing to be bound by theory, it is believed that the DZ-1-Lys-DOTA-¹⁷⁷Lu complex may be particularly advantageous for smaller tumors or metastases, e.g. tumors of a width/height and length of about 5 mm or less, 2 mm or less, 1 mm or less, or about 0.5 mm or less. DZ-DOTA-¹⁷⁷Lu may provide higher absorbed doses at high stability of the complex, which in combination may help protect healthy tissues from absorbing dissociated radiometals.

Surprisingly, it has been shown that DZ-1-Lys-DOTA-6Ga, DZ-DOTA-¹⁷⁷Lu, and DZ-1-Lys-DOTA-⁹⁰Y form stable complexes that remain stable in serum for a sufficient time for clinical application, as shown in examples 11-17 herein-below. DZ-1-Lys-DOTA-⁶⁸Ga in particular has an excellent stability while DZ-DOTA-¹⁷⁷Lu (half-life about 700 min) and DZ-1-Lys-DOTA-⁹⁰Y (half-life about 240 min) have a moderate but sufficient stability and efficient tumor retention for effective cancer treatment.

Without wishing to be bound by theory, it is believed that the DZ-1-Lys-DOTA-⁹⁰Y complex may be particularly advantageous due to its good serum stability and its long penetration range while at the same time allowing sufficiently fast clearance from blood circulation, and can thus advantageously be used especially for larger tumors, e.g. of a length, height and/or width of about 1 mm or more, about 2 mm or more, 5 mm or more, 10 mm or more, or 12 mm or more.

Without wishing to be bound by theory, it is believed that the DZ-1-Lys-DOTA-⁶⁸Ga complex may be particularly advantageous for its high stability in serum, e.g. as compared to a Cu-64 complex.

In embodiments, provided are methods for image-guided radiotherapy wherein for therapy, the radiometal is selected from the group consisting of Lutetium-177, Yttrium-90, and for enhanced imaging, the radiometal is Gallium-68. Surprisingly, Lu-177, Y-90 and Ga-68 all appear to be more stable in serum compared to previously known complexes, e.g. compared to the same complex with Copper (Cu-64). At the same time these complexes appear to be at least similarly effective in tumor targeting, tumor shrinking or cancer cell killing.

In embodiments, provided are radiometal complexes of a DOTA chelator conjugated to DZ-1, a heptamethine cyanine dye (HMCD), via a lysine linker (see formula I shown herein below), compositions comprising these complexes, or compositions for making these complexes, and the use of such compositions or complexes for the radiotherapy of cancer. The present invention also relates to methods of making the conjugates and complexes, pharmaceutical compositions including the complexes, methods of using the conjugates, complexes or pharmaceutical compositions, methods of imaging and/or radiotherapy of cancer cells, tissues, tumors, and/or their metastases, kits for imaging and/or radiotherapy, and the like. In addition, the present disclosure includes compositions used in and methods relating to non-invasive imaging, in particular PET or SPECT imaging, of the complexes in vivo. The HMCD-DOTA radiometal complexes may be used for radio therapy of cancer cells and tissues, including of solid tumors. These compounds may have various advantages which may including an improved penetration of tumors.

In embodiments, provided is a DZ1-Lys-DOTA radiometal complex which comprises a heptamethine carbocyanine dye (HMCD) moiety conjugated with DOTA via a lysine linker and comprising a complexed radiometal M, wherein M is selected from Lu-177, Y-90, and Ga-68, as shown in FI below:

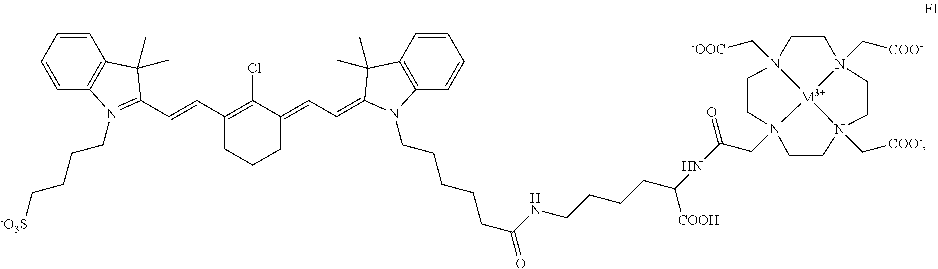

In an embodiment, provided is a DZ-1-Lys-DOTA conjugate for forming a radiometal complex, as shown in FII below:

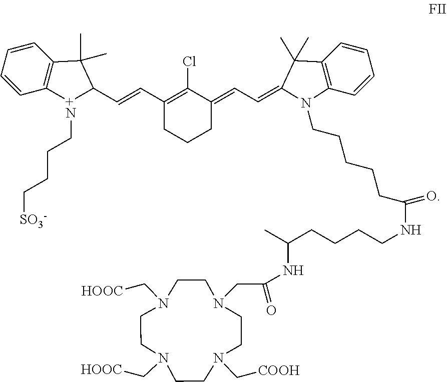

It is believed that the complexes described herein may provide one or more advantages including very stable radiometal complexes that can provide an effective cancer therapy with low toxicity and good tumor penetration even of solid tumors. In particular, the complexes may be suitable for therapeutic applications rather than mere imaging, and may provide one or more of the following advantages: a more accurate quantitative analysis via PET or SPECT probe and/or optionally near infrared fluorescence (NIRF), a higher contrast between tumor and noncancerous tissues, a higher sensitivity, a better selectivity for cancer cells, in particular in vivo, a rapid steady state distribution in the body and organs including heart, liver, lungs and kidney, rapid tumor targeting, a uniform distribution in tumor tissues, a lack of persistence of radioactivity in one or more organs including kidney and/or liver. Further, these complexes may advantageously be more easily formed and/or at lower cost, may be easily labelled with a radiometal, may form stable complexes with one or more radiometal, may have high complex formation rates, a high radiochemical yield, a good labelling efficiency with one or more radiometal, a good solubility in aqueous solutions, a high stability in aqueous solutions, a high stability at physiological pH of about 7.4 (e.g. 7.2-7.6), a high stability in vivo, a low rate of dissociation of the radiometal from the complex, in particular in vivo, a good biodistribution, sufficient targeting of the complex to cancer tissues, fast cancer-specific targeting, sufficient targeting for tumor and/or metastases, sufficient targeting for larger tumors, a low organ accumulation (e.g. kidney, spleen, liver, heart), no or reduced radioactive exposure of tissues and organs, a rapid clearance rate from important or sensitive organs (e.g. from blood, liver, kidneys and other organs) after administration, sufficient tissue and tumor penetration, sufficient penetration of encapsulated tissues such as organs including dense tissues including tumor tissues, in particular calcified tumor tissues, improved and/or longer retention in the tumor.

Radiochemical yield for the isotopes of a specified element is the yield of a radiochemical separation expressed as a fraction or percentage of the activity originally present. Also called the recovery. In radiation chemistry, the number of species transformed by radiation per eV of absorbed energy: represented by the symbol G, the G-value. Radiochemical yield may be determined as will be apparent to a person of ordinary skill.

It is believed that the high stability of the complexes, including in serum, see examples herein-below, in addition to the combination with DOTA and the particular radiometal, may contribute to one or more advantages as described herein.

Complexes as described herein may be able to penetrate more easily and more deeply into a tumor. These radiopharmaceuticals may thus be better suited for solid tumors, in particular denser and/or larger and/or partially calcified tumors. This may particularly be applicable to DZ-1-Lys-DOTA-⁹⁰Y, which is believed to provide improved therapy for larger tumors of a volume of e.g. 5-15 cm³ or more.

Complexes as described herein may be able to provide blood-brain-barrier (BBB) penetration and thus may be used for treatment of brain tumors and brain metastases.

Complexes as described herein may be able to provide higher tumor accumulation ratios and/or a fast blood clearance. Distribution in animals may show a favorable time-dependent clearance in their organs concomitant with specific accumulation in tumors (e.g. xenograft tumors in mice, see e.g. example 3 and 7).

Complexes as described herein may be able to provide an improved percentage and/or speed of tumor uptake, e.g. as measured by % of injected dose (ID) of FI (20 μg/kg) and the tumor-to-blood ratio at points in time (e.g. at 4, 8, 16 and/or 24 hours), as will be apparent to a person of ordinary skill.

Complexes as described herein may be able to provide increased dosing options and less accumulation and/or improved organ clearance from organs including one or more of kidneys, liver and heart. Organs such as the kidneys often are dose-limiting organs in radiotherapy.

Absorbed doses may be measured as will be apparent to a person of ordinary skill, and may be e.g. about 15 Grays (Gy) or less, e.g. 10, 5, 2, 1 Gy or less.

Complexes as described herein may be able to provide a reduced absorbed dose to organs and/or soft tissues such as kidney, spleen, liver, and/or hematological toxicity (e.g. thrombocytopenia, neutropenia).

Complexes as described herein may be able to provide improved effectiveness against cancer at lower amounts of radioactivity. For example, less than 50 μCi, e.g. less than 20, 20, 5 or 2 μCi may be needed.

Complexes as described herein may be able to provide reduced toxicity at doses sufficient to treat cancer and/or significantly reduce tumor volume (e.g. by at least 10, 20, 30, 40, 50, 60, 70, 80, 90 or 100%); thus a reduced LD50 and/or organ toxicity e.g. reduced kidney, spleen, liver, bone marrow, brain, heart or lung toxicity may be provided, and/or the complexes may allow to avoid or improve weight loss which often occurs during and after therapy.

Complexes as described herein may be able to provide reduced toxicity at doses sufficient to treat cancer and/or significantly reduce tumor volume while improving one or more of pain scores, bone pain scores, and median survival.

Complexes as described herein may be able to provide sufficient anti-cancer/anti-tumor effects at reduced total dose, for example less than 250 kBq/kg, e.g. less than 200, 150, 50, and 25 kBq/kg.

Complexes as described herein may be able to provide sufficient effectiveness to treat cancer and/or significantly reduce tumor volume while avoiding one or more side effects such as pain, dizziness, nausea, effects on the digestive tract, stomach pain, constipation, diarrhea, hair loss.

In embodiments, radiometal complexes can provide therapy through the decay of the radiometals which deposits radiation energy in or near the target cells or tissues of a dose that kills the target cells or tissues, in particular tumor cells or tissues, or metastases thereof. In particular, Lu-177 and Y-90 are β-emitting radiometal which emit positrons. Lu-177, Y-90 and/or Ga-68 may be able to form stable complexes with the DZ-1-Lys-DOTA conjugates described herein that are stable in serum, such therapeutic complexes can provide an advantageous therapeutic effect and be used for radiotherapy of cancer.

In embodiments, radiotherapy as described herein may be improved by pairing such therapy with Positron Emission Tomography (PET) or SPECT, in particular by using Ga-68 to improve detection and/or localization primary tumors, secondary and further tumors, and/or metastases, thus providing an improved image-guided therapy (see examples herein-below, in particular example 3). PET detects positron-emitters, and may optionally be combined with Computerized Tomography (CT) imaging, i.e. PET/CT. In CT, x-ray scans are taken from different angles with the various slices arranged in 3D by a computer which may be used as a map to overlay with signals detected by PET.

In embodiments, radiotherapy may be performed internally, i.e. a source of radiation may be provided inside the subject's body for cancer therapy. Internal radiation therapy may be performed systemically, thus the treatment travels in the blood to tissues throughout the body to kill cancer cells in a targeted fashion trough the radiation the cells in proximity to the radiometal are exposed to. In embodiments, treatment may be through systemic or localized/site-specific administration, such as oral or intravenous injection for systemic administration, or localized injection or deposits, e.g. through use of seeds in brachytherapy of accessible tumors, as will be apparent to a person of ordinary skill. Using the stable complexes provided, the radiometals can be delivered to tumors and metastases in a targeted fashion.

In embodiments, one or more radiometal complex may be administered in a coordinated administration protocol. In particular, a complex selected from Lu-177, Y-90 may be administered with a Ga-68 complex, e.g. sequentially, with Ga-68 being administered before and/or after, or simultaneously.

Without wishing to be bound by theory, it is believed that the DZ-1-Lys-DOTA form complexes of high thermodynamic stability and kinetic inertness with Lu-177, Y-90 and Ga-68, and will rapidly, quantitatively and stably coordinate with the radiometal at room temperature (at about 20 DEG C, or e.g. at less than 25, 30, 35, 40, 45, 50, 55, 60, 65, 70 or 75 DEG C), near neutral pH (at about 7, e.g. about 5 or more, about 6 or more, about 8 or less, about 7.5 or less), and at a low DZ-1-Lys-DOTA concentration, e.g. 0.1-100 micromolar or nanomolar, e.g. about 1 to about 20 micromolar, e.g. about 1 to about 10 micromolar, e.g. about 5-8 micromolar, allowing to easily provide radiopharmaceutical formulations.

Complexes as described herein comprising Gallium-68 (⁶⁸Ga) may provide superior imaging during detection including e.g. PET, in particular one or more of better signal-noise ratio, stronger signal, and better resolution.

Complexes as described herein comprising Lutetium-177 (¹⁷⁷Lu, Lu-177) may be suitable for tumors of all sizes with effects including tumor shrinkage, and may be particularly effective to destroy small tumors or metastases of a volume of less than e.g. about 5 to about 0.1 cm³ or less, e.g. about 5 cm³ or less, about 4 cm³ or less, about 3 cm³ or less, about 2 cm³ or less, about 1 cm³ or less, about 0.5 cm³ or less, about 0.1 cm³ or less. Where not otherwise indicated, tumor volumes apply to human patients typically having a weight of about 140 to 200 lb, e.g. about 170 lb. In non-human subjects tumor volumes will be less or more depending on body weight.

Advantages may include a significant reduction or removal of metastases, higher absorbed doses, and/or a lower tissue penetration range.

Complexes as described herein comprising Yttrium (⁹⁰Y, Y-90) are suitable for tumors of all sizes with effects including tumor shrinkage. Advantages may include a significant reduction or removal of metastases, higher absorbed doses, and/or a lower tissue penetration range.

In embodiments, Positron Emission Tomography (PET) may be used to provide data on radiometal distribution within target tissues by detection of gamma photons resulting from the decay of the radiometals. High spatial resolution of commonly available PET scanners allows to visually map radiometal decay events and thus provide an image which reflects the distribution of a radiometal in the body after administration of a radiometal complex. Such images provide anatomic and functional information to aid medical diagnosis and assist to track progress and allow adjustment of radiotherapy.

In embodiments, cancer as referred to herein includes precancerous and cancerous cells or tissues, tumors and their metastases, primary and secondary tumors. The conjugates and methods described herein may be particularly advantageous for use in brain cancers, brain tumors and their metastases. Without wishing to be bound by theory it is believed that DOTA, in combination with the radiometals as described, namely Lutetium-177, Yttrium-90 and Gallium-68, and when conjugated via a lysine linker, allows the radioactive conjugates to pass across the blood-brain barrier and allow imaging and therapy throughout the brain, including tumors located deep within the brain structure that often are not or not efficiently accessible by other methods and may be inoperable.

In embodiments, the cancer treated by the complexes may be selected from the group comprising: brain cancer, prostate cancer, lung cancer, Non-small-cell lung carcinoma (NSCLC), small-cell lung carcinoma (SCLC), pancreatic cancer, kidney cancer, lymphoma, colorectal cancer, skin cancer, HCC cancer, and breast cancer, squamous-cell carcinoma of the lung, anal cancers, epithelial tumors of the head and neck, bone cancer, carcinoma of the cervix, skin cancer, melanoma, hematopoietic cancers, lymphoma, and myeloma, or metastases of any thereof, including e.g., without limitation, metastases occurring in the brain, the bone, or other organs, brain tumors or their metastases, brain tumors and their brain, bone, lung or other organ metastases, bone tumors or their brain, bone, lung or other metastases, prostate tumors or their brain, bone, lung or other metastases, prostate tumors and their brain, bone, lung or other metastases, lung tumors and their brain, bone, lung or other metastases, and others.

In embodiments, the cancer may be a central nervous system (CNS) or brain tumor, or metastasis thereof, selected from the group comprising: acoustic neuroma, astrocytoma, chordoma, CNS lymphoma, craniopharyngioma, glioma, glioblastoma, medulloblastoma, meningioma, oligodendroglioma, pituitary tumors, primitive neuroectodermal tumor, Schwannoma, brain stem glioma, ependymoma, juvenile pilocytic astrocytoma, optic nerve glioma, pineal tumor, rhabdoid tumor, adult Low-Grade (WHO Grade I or II) Glioma/Pilocytic, Infiltrative Supratentorial Oligodendroglioma, Anaplastic Gliomas/Glioblastoma, Adult Intracranial Ependymoma, Adult Medulloblastoma, Primary CNS Lymphoma, Primary Spinal Cord Tumors, Limited Brain Metastases, Extensive Brain Metastases, Leptomeningeal Metastases, and Metastatic Spine Tumors.

In embodiments, the cancer may be a Non-small-cell lung carcinoma selected from a Squamous-cell carcinoma, Adenocarcinoma (Mucinous cystadenocarcinoma), Large-cell lung carcinoma, Rhabdoid carcinoma, Sarcomatoid carcinoma, Carcinoid, Salivary gland-like carcinoma, Adenosquamous carcinoma, Papillary adenocarcinoma, and Giant-cell carcinoma.

Alternatively, the cancer may be a small-cell lung carcinoma, including a Combined small-cell carcinoma. Alternatively, the cancer may be a non-carcinoma of the lung, including a Sarcoma, Lymphoma, Immature teratoma, and Melanoma.

In embodiments, a pharmaceutical composition comprising a radiometal complex, or for forming such complexes, is provided. The pharmaceutical composition may be for human or for veterinary use, and comprise one or more conjugate or complex of the invention (or a salt, solvate, metabolite, or derivative thereof) with one or more pharmaceutically acceptable carrier and/or one or more excipient and/or one or more active. The one or more carrier, excipient and/or active may be selected for compatibility with the other ingredients of the formulation and not unduly deleterious to the recipient thereof. Such carriers are known in the art and may be selected as will be apparent to a person of ordinary skill in the art.

In embodiments, routes of administration for the compounds and pharmaceutical compositions include, but are not limited to: oral (e.g. in pill form), intravenous (i.e. injected into a subject's vein), interstitially (i.e. inserted into a space in the body), intraperitoneal, subcutaneous, or intramuscular, and/or by brachytherapy (insertion of radioactive implants or seeds directly into the affected tissue, e.g. into or near a tumor location). Administration may be systemic (e.g. via blood circulation) or regional (e.g. localized to a particular organ of the body or part thereof). In some embodiments, the pharmaceutical compositions of the invention contain a pharmaceutically acceptable excipient suitable for rendering the compound or mixture administrable via the above routes of administration.

In embodiments, the active ingredients can be admixed or compounded with a conventional, pharmaceutically acceptable excipient or carrier. A mode of administration, vehicle, excipient or carrier should generally be substantially inert with respect to the active agent, as will be understood by those of ordinary skill in the art. Illustrative of such methods, vehicles, excipients, and carriers are those described, for example, in Remington: The Science and Practice of Pharmacy (2020), ISBN-10: 0128200073, or in Handbook of Pharmaceutical Excipients, Ninth edition (2020), ISBN-10: 0857113755, the disclosures of which is incorporated herein by reference. The excipient must be “acceptable” in the sense of being compatible with the other ingredients of the formulation and not deleterious to the recipient thereof.

In embodiments, the pharmaceutical formulations may be conveniently made available in a unit dosage form by any of the methods generally known in the pharmaceutical arts. Generally speaking, such methods of preparation comprise presenting the formulation in a suitable form for delivery, e.g., forming an aqueous suspension. The dosage form may optionally comprise one or more adjuvant or accessory pharmaceutical ingredient for use in the formulation, such as mixtures, buffers, and solubility enhancers.

According to an embodiment of the present invention, parenteral dosage forms (i.e. that bypass the GI tract) of the pharmaceutical formulations include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, administration DUROS®-type dosage forms, and dose-dumping.

In embodiments, suitable vehicles that can be used to provide parenteral dosage forms of the compounds of the invention include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a compound of the invention as disclosed herein can also be incorporated into the parenteral dosage forms of the disclosure, including conventional and controlled-release parenteral dosage forms.

In embodiments, formulations for parenteral administration include aqueous and non-aqueous sterile injection solutions, which may further contain additional agents, such as anti-oxidants, buffers, bacteriostats, and solutes, which render the formulations isotonic with the blood of the intended recipient. The formulations may include aqueous and non-aqueous sterile suspensions, which contain suspending agents and thickening agents.

In embodiments, injectable preparations, for example, sterile injectable aqueous or oleaginous suspensions can be formulated according to the known art using suitable dispersing or wetting agents and suspending agents. The sterile injectable preparation can also be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides.

In addition, fatty acids such as oleic acid are used in the preparation of injectables.

In embodiments, forms suitable for oral administration include tablets, troches, capsules, elixirs, suspensions, syrups, wafers, or the like prepared by art recognized procedures. The amount of active compound in such therapeutically useful compositions or preparations is such that a suitable dosage will be obtained. A syrup formulation will generally consist of a suspension or solution of the compound or salt in a liquid carrier, for example, ethanol, glycerine or water, with a flavoring or coloring agent.

In embodiments, solid dosage forms for oral administration include capsules, tablets, pills, powders, and granules. In such solid dosage forms, the active compound is mixed with at least one inert, and/or a) fillers or extenders such as starches, lactose, sucrose, glucose, mannitol, and silicic acid, b) binders such as, for example, carboxymethylcelhdose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form can also comprise buffering agents.

In embodiments, solid compositions of a similar type can be employed as fillers in soft and hardfilled gelatin capsules using such excipients as lactose or milk sugar as well as high molecular weight polyethylene glycols, and the like. The solid dosage forms of tablets, dragees, capsules, pills, and granules can be prepared with coatings and shells such as enteric coatings and other coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type can also be employed as fillers in soft and hard-filled gelatin capsules using such excipients as lactose or milk sugar as well as high molecular weight polyethylene glycols, and the like.

In embodiments, the active compounds conjugates or complexes can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound can be admixed with at least one inert diluent such as sucrose, lactose and starch. Such dosage forms can also comprise, as in normal practice, additional substances other than inert diluents, e.g., tableting lubricants and other tableting aids such as magnesium stearate and microcrystalline cellulose. In the case of capsules, tablets and pills, the dosage forms can also comprise buffering agents. They can optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

In embodiments, the active compounds, conjugates or complexes can be present in form of salts, which may be particularly suitable for use in the treatment of cancer. The salts of the present invention may be administered to the patient in a variety of forms, depending on the route of administration, the salt involved, and the cancer being treated. For example, an aqueous composition or suspension of the salts may be administered by injection, or in the form of a pharmaceutical matrix by injection or surgical implantation, at a desired site. The particular technique employed for administering the matrix may depend, for example, on the shape and dimensions of the involved matrix. In some embodiments, the salt is introduced substantially homogeneously in a tumor to minimize the occurrence in the tumor of cold (untreated) areas. In certain embodiments, the salt is administered in combination with a pharmaceutically acceptable carrier. “Pharmaceutically acceptable carrier” refers to an excipient that can be included in the compositions of the invention and that causes no significant adverse toxicological effects to the patient. A wide variety of pharmaceutically acceptable carriers or excipients are available and can be combined with the present salts, as will be apparent to one of ordinary skill in the art.

According to an embodiment of the present invention, effective amounts, toxicity, and therapeutic efficacy of the active compounds conjugates or complexes can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. In some embodiments, compositions and methods exhibit large therapeutic indices. A therapeutically effective dose can be estimated initially from cell culture assays. Also, a dose can be formulated in animal models to achieve a circulating plasma concentration range that includes the IC50 (i.e., the concentration of the compound of the invention, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

In embodiments, the dosage of a pharmaceutical formulation as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule/regimen can vary, e.g. once a week, daily, or in particular predetermined intervals, depending on a number of clinical factors, such as the subject's sensitivity to each of the active compounds.

In embodiments, an effective dose of a composition comprising radiometal complex can be administered to a patient once. Alternatively, an effective dose of a composition comprising a radiometal complex can be administered to a patient repeatedly. The radiometal complex can be administered over a period of time, such as over a 5 to 60 minute period, e.g. about 30 minutes. If warranted, the administration can be repeated, for example, on a regular basis, such as hourly, daily, bi-weekly or weekly, e.g. in a suitable time interval of e.g. about 6, 12, 24, 48 or 72 hours. In some instances, after an initial treatment regimen, the treatments can be administered on a less frequent basis. For example, after an initial administration, administration can be repeated once per week, month, six months or a year or longer.

In embodiments, the amount of the active compound, conjugate or complex in the pharmaceutical composition can be based on weight, moles, or volume. In some embodiments, the pharmaceutical composition comprises at least 0.0001%, 0.1%, 0.5% 1%, 2%, 3%, 4%, 5% or 10% of the active. In some embodiments, the pharmaceutical composition comprises 0.01%-99% of the active, e.g. 0.05%-90%, 0.1%-85%, 0.5%-80%, 1%-75%, 2%-70%, 3%-65%, 4%-60% or 5%-50% of the active.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the compound of the invention.

In embodiments, in addition to treating cancer (pre-cancerous or cancerous cells and tumors) in a subject in need thereof, such cancer is identified, imaged and/or localized. The method may comprise providing a radiometal complex; administering the complex to a subject; and optionally performing imaging, e.g. PET imaging, to detect the emitter released. This allows to visually follow tumor growth and/or shrinkage, e.g. to confirm or personalize an optimized dosage, and/or determine the location of tumor(s) and/or metastases. In various embodiments, imaging may be performed, for example, about 6 to 48 hours post administration. Imaging may be performed in comparison to normal tissue/cells to determine uptake rates differing in cancer/tumor tissues versus normal tissues.

In embodiments, in situ pharmacokinetic and pharmacodynamic analyses in a tumor or normal cell or tissue may be performed. The method can comprise providing the complex; contacting it with the cancer cells, tumor, or normal cell or tissue; and imaging the cancer cells, tumor, or normal cell or tissue, followed by pharmacokinetic and/or pharmacodynamics analyses, e.g. determining the signal (including radioactivity and/or NIR fluorescence, and changes thereof, at one or more point of time over time, e.g. before and after administration, and one or more times after administration.

In embodiments, a kit for providing or making a radiopharmaceutical preparation comprising a radiometal complex is provided. In general a vial containing the nonradiometal components of a radiopharmaceutical preparation, usually in the form of a sterilized, validated product to which the appropriate radiometal is added or in which the appropriate radiometal is diluted before use. One or more kit component may be provided in form of an optionally buffered solution, or may be provided for dissolution in an optionally buffered solution. For example, the kit may be a single or multidose vial, and the radiopharmaceutical preparation may require additional steps, e.g., without limitation, boiling, heating, filtration and/or buffering.

Radiopharmaceutical preparations derived from kits generally are intended for immediate use after preparation, e.g. within 6-24 hours, e.g. within about 12 hours of preparation.

The kit can also further comprise conventional kit components, such as needles for use in injecting the compositions, one or more vials for mixing the composition components, and the like, as are apparent to those of ordinary skill. In addition, instructions, either as inserts or as labels, indicating quantities of the components, guidelines for mixing the components, and protocols for administration, can be included in the kits.

The concentration of the complexes employed in the pharmaceutical compositions and/or the amount administered to a patient or subject may vary and depends upon a variety of factors including, for example, the particular complex and/or pharmaceutically acceptable carrier employed, the particular disease being treated, the extent of the disease, the size and weight of the patient, and the like. Typically, the complex may be employed in the pharmaceutical compositions, and the compositions may be administered to a patient to provide initially lower levels of radiation dosages which may be increased until the desired therapeutic effect is achieved. Generally speaking, the complexes may be employed in pharmaceutical compositions which comprise an aqueous carrier to provide a concentration of absolute radioactivity which may range from about 4 MBq per milliliter (ml) (about 0.1 mCi/ml) or less to about 370 MBq/ml (about 10 mCi/ml), and all combinations and subcombinations of ranges therein. In embodiments, the concentration of the complex in the pharmaceutical compositions may be from about 37 MBq/ml (about 1 mCi/ml) to about 370 MBq/ml (about 10 mCi/ml). In addition, the compositions may be administered to a patient to provide a radiation dose which may range from about 1 KSv (about 1×105 Rem) to about 74 KSv (about 7.4 MRem), and all combinations and subcombinations of ranges therein. In embodiments, the compositions may be administered to a patient to provide a radiation dose of from about 7.4 KSv (about 7.4×105 Rem) to about 74 KSv (about 7.4 MRem). Such amounts are referred to herein as effective amounts or therapeutically effective amounts. In embodiments, the pharmaceutically acceptable carrier may further comprise a thickening agent.

Thickening agent refers to any of a variety of generally hydrophilic materials which, when incorporated in the present compositions, may act as viscosity modifying agents, emulsifying and/or solubilizing agents, suspending agents, and/or tonicity raising agents.

Thickening agents which may be suitable for use in the present radiopharmaceutical compositions include, for example, gelatins, starches, gums, pectin, casein and phycocolloids, including carrageenan, algin and agar, semi-synthetic cellulose derivatives, polyvinyl alcohol and carboxyvinylates, and bentonite, silicates and colloidal silica. Other thickening agents would be apparent to one of ordinary skill.

The concentration of thickening agent may range from about 0.1 to about 500 milligrams (mg) per ml of pharmaceutical composition. In certain embodiments, the concentration of thickening agent may be from about 1 to about 400 mg/ml, e.g. from about 5 to about 300 mg/ml, e.g. from about 10 to about 200 mg/ml, e.g. from about 20 to about 100 mg/ml, or e.g. from about 25 to about 50 mg/ml. Compositions which may be prepared from the complexes, pharmaceutically acceptable carriers and optional thickening agents include, for example, suspensions, emulsions, and dispersions. In some embodiments, the complexes can be formulated and administered to a patient as a suspension.

Suspension may refer to a mixture, dispersion or emulsion of finely divided colloidal particles in a liquid. Suspensions may be obtained, for example, by combining the complexes with an inert solid support material. Particulate support materials which may be suitable for use as an inert solid support in the compositions of the present invention include, for example, materials derived from carbon, including those forms of carbon typically referred to as carbon black (lampblack) and/or activated carbon, as well as finely powdered oxides, Kieselguhr, and diatomaceous earth. In some embodiments, the support material comprises carbon black or activated carbon. The size of the particles of the particulate support material may vary and depends, for example, on the particular support material, complex, thickening agent, and the like, employed and may comprise particles ranging in size, for example, from about 0.1 micrometer (mm) to about 50 mm, e.g. the particle size may be from about 0.5 to about 25 mm, e.g. from about 1 to about 10 mm, e.g. from about 2 to about 5 mm.

Delivery of a therapeutically effective activity of a complex can be obtained via administration of a pharmaceutical composition comprising a therapeutically effective activity or dose of the complex, i.e. in a concentration that is sufficient to elicit the desired therapeutic or imaging effect according to the methods described herein. A therapeutically effective activity may be an activity effective to treat cancer, such as inhibiting or slowing growth of cancerous or precancerous tissue, or lowering the survival rate of cancer or pre-cancerous cells. The therapeutically effective dosage will vary somewhat patient to patient, and will depend upon the condition of the patient and the route of delivery. The effective activity of any particular compound would be expected to vary according to the weight, sex, age, and medical history of the subject. Other factors which influence the effective dose can include, but are not limited to, the severity of the patient's condition, the disease or disorder being treated, the stability of the complex, and, if appropriate, any additional antineoplastic therapeutic agent being administered with the complex. Methods to determine efficacy and dosage are known to those of ordinary skill.

### EXEMPLARY ILLUSTRATIVE EMBODIMENTS

### Example 1—Synthesis of DZ-1-Lys-DOTA Conjugate Radiometal Complexes

The synthesis of the DZ-1 dye and its derivatives has been previously described, e.g. in U.S. Pat. No. 10,307,489, which is hereby incorporated herein in its entirety, and may be performed, for example, as described in detail in example 1a below. The conjugate may then be complexed with radiometals as described in example 1b. The following chemicals and reagents may be used.

DOTA, also known as 1,4,7,10-Tetraazacyclododecane-1,4,7,10-tetraacetic acid, DOTA-tris (t-Bu ester), also known as 1,4,7,10-Tetraazacyclododecane-1,4,7-tris-tert-butyl acetate-10-acetic acid, may be purchased from Macrocyclics (Arlington Heights, IL). DOTA and all other chemicals mentioned herein, e.g. in the reaction schemes as shown, may also be purchased from various standard sources, for example VWR International (Radnor, PA) or Thermo Fisher Scientific (Waltham, MA), as will be apparent to a person of ordinary skill. Deionized ultrapure water (18.2 MQ) may be used for making solutions which may be obtained from Milli-Q Direct Ultrapure Water System from Millipore (Billerica, MA, USA). Analytical reversed-phase (RP) high-performance liquid chromatography (HPLC) may be performed on an Agilent system, e.g. with a 1260 Infinity Diode-Array Detector, e.g. with an Apollo C18 RP column (5 μm, 150×4.6 mm). For example, the mobile phase may change from 60% solvent A (e.g. 0.1% trifluoroacetic acid in 80% water) and 40% solvent B (e.g. 0.1% trifluoroacetic acid in 80% aqueous acetonitrile) to 100% solvent B over a period of e.g. about 30 min at a flow rate of about 1 ml/min monitoring at e.g. about 254 and about 780 nm). Electrospray Ionization Mass Spectrometry (ESI-MS) analysis may be performed on the synthesized compounds, e.g. on a Thermo LTQ Orbitrap Elite mass spectrometer system.

Example 1a—Synthesis of a DZ-1-Lys-DOTA conjugate

Scheme 1 shown below illustrates the first steps of conjugate synthesis forming a DZ-1-Lys-DOTA conjugate, compound 4, also referred to herein as “DZ-1”; alternative routes of synthesis will be apparent to a person of ordinary skill. DZ-1 may then be conjugated to the lysine linker (shown in scheme 2 below), and then further conjugated to DOTA (shown in schemes 3 and scheme 4 (or alternatively to scheme 4 in scheme 5) below.

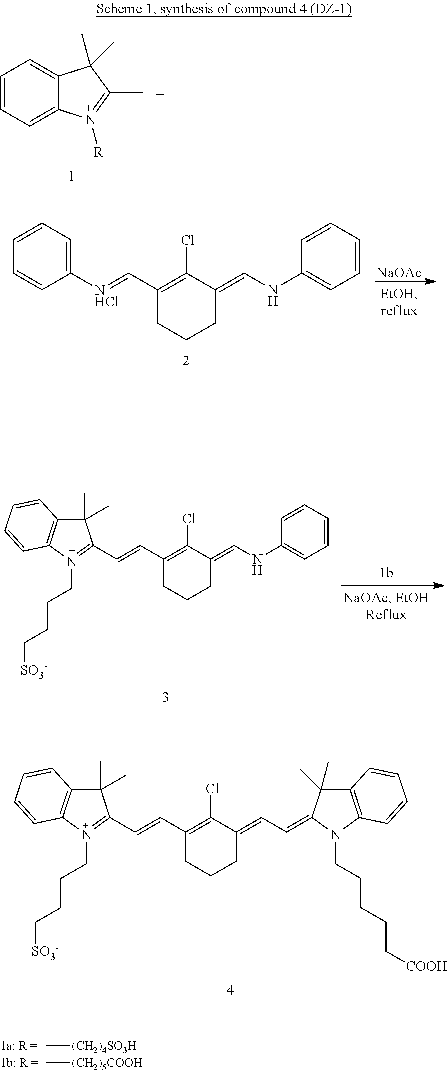

Synthesis of compound 1a (compare scheme 1 above): The mixture 2, 3, 3-trimethylindolenine (5 g, 31.4 mmol) and 1,4-butane sultone (5.1 g, 37.7 mmol) may be heated with stirring at 120° C. under argon for 5 h. The resulted reaction mixture may be cooled to RT (e.g. about 20° C.) and the solid may be dissolved in a sufficient volume of solvent, e.g. and organic solvent, e.g. about 50 ml of methanol. Ethyl ether, e.g. about 200 ml, may be added to the methanol solution for precipitation, and the precipitate may be collected and washed with a sufficient volume a sufficient number of times of ethyl acetate (e.g. 15 ml, three times) and dried, e.g. under vacuum, to afford desired product 6.8 g (yield 73%) as a white solid.

Synthesis of compound 1b (compare scheme 1 above): To 6-Bromohexanoic acid (2.5 g, 13.0 mmol) may be added 2, 3, 3-trimethylindolenine (2.5 g, 15.7 mmol). The reaction mixture may be heated with stirring at a sufficiently elevated temperature, e.g. at about 110° C. under a protective gas such as argon for 8 h. The resulting dark red solid may be dissolved in 50 ml of methanol. Ethyl ether 150 ml may be added. The precipitate may be filtered and washed a sufficient number of times and volume e.g. with ether (e.g. 15 ml, three times) followed by a sufficient number of volume and times washing with acetone (e.g. 15 ml, three times). The product obtained is a white solid (2.7 g, 58%).

Synthesis of compound 3 (compare scheme 1 above): To the mixture of 1a (2 g, 6.78 mmol) and compound 2 (3 g, 8.36 mmol) in EtOH (100 ml) may be added CH₃COONa (0.28 g, 3.39 mmol), the resulted mixture may be heated at a sufficient temperature and duration, e.g. about 60° C. for about 18 hours. The precipitate may be filtered and sufficiently washed with cold ethanol (e.g. 20 ml, three times). The product may be dried under vacuum to afford desired product 3 as a dark blue solid (2.1 g, yield 58%). Mass spectrum (ESI) 525.19 [M+H]⁺.

Synthesis of compound 4, a heptamethine cyanine dye, also referred to herein as DZ-1 (compare scheme 1 above): To the mixture of 1b (0.67 g, 1.9 mmol) and compound 3 (1.0 g, 1.9 mmol) in EtOH (20 ml) may be added CH₃COONa (156 mg, 1.9 mmol), the resulting solution may be heated to reflux for a sufficient time, e.g. 3 h. The mixture may then be subjected to precipitation, e.g. kit may be poured into 100 ml of ice water. The solid may be filtered and crystallized from methanol-water to afford desired product 4 as a dark green solid (0.99 g, yield 74%). Mass spectrum (ESI) 705.31 [M+H]⁺.

Scheme 2 below shows a synthesis of a dye conjugated to a suitable linker moiety, here of DZ-1-Lysine:

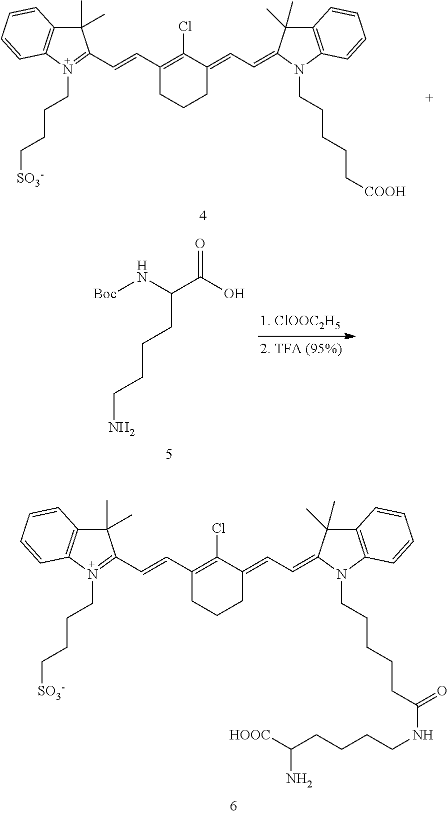

Synthesis of DZ-1-Lysine (compare scheme 2 above): DZ-1 4 (200 mg, 0.28 mmol) may be dissolved in 5 ml anhydrous CH₂C2. Ethyl chloroformate (ClCOOC₂HS) (46 mg, 0.42 mmol) and triethylamine (57 mg, 0.57 mmol) may be added. The mixture may be stirred for sufficient duration, e.g. about 2 hours, then N-α-Boc-Lysine 5 (70 mg, 0.28 mmol) in a sufficient volume, e.g. 2 ml of DMF, may be added, and stirred for an additional sufficient duration, e.g. about 2 hours at room temperature (RT). The crude materials may be precipitated in cold diethyl ether (40 ml). Centrifugation for sufficient duration and rpm, e.g. about 5 min at about 3500 rpm, enabled recovery of the pellet which may be purified, e.g. by C18-RP silica chromatography elution with acetonitrile in aqueous NH₄HCO₃ solution (20 mM) to afford desired product DZ-1-(N-α-Boc)-Lysine as a dark green solid 109 mg (42%). DZ-1-(N-α-Boc)-Lysine may be dissolved in TFA (95%) 5 ml and the mixture may be sufficiently stirred, e.g. about 3 h at ambient temperature, e.g. at RT (about 20 degree centigrade). A sufficient amount of ethyl ether, e.g. 40 ml, may be added. The suspension may be centrifuged for sufficient time to achieve separation, allowing to decant the ether. The product may be dried, e.g. by placing it under high vacuum for sufficient duration, e.g. overnight. The resulting product 6 may be used without further purification for the steps described below.

Schemes 3 and 4 (or alternatively scheme 5 show a synthesis of an activated chelator compound (here: DOTA-sulfo-NHS, compare scheme 3), which may then be conjugated to the DZ-1-Lys compound (compare scheme 4 for a first method, or alternatively, scheme 5, for a second method).

Scheme 3 below shows a synthesis of an activated chelator compound, here: DOTA-sulfo-NHS:

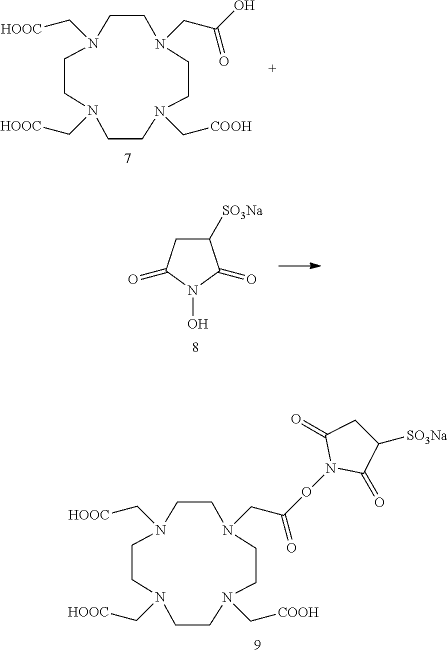

Synthesis of DOTA-Sulfo-NHS (compare scheme 3 above): DOTA 7 may be activated by EDC at a suitable pH, e.g. pH of about 5.5 for a suitable duration, e.g. about 30 min (4° C.), with suitable molar ratio, e.g. a molar ratio of DOTA:EDC:Sulfo-NHS) of about 10:5:4.

DOTA (24.2 mg, 48 μmol) may be dissolved in a suitable amount and solvent, e.g. in about 500 μL of water and about 4.6 mg of EDC (24 μmol) dissolved in a suitable volume of water, e.g. about 130 μl of water, may be mixed, and 0.1 N NaOH may be added to adjust the pH to a suitable degree, e.g. to about pH 5. Sulfo-NHS (4.2 mg, 19.2 μmol) may be then added to the stirring mixture upon cooling, e.g. in an ice-bath, and 0.1 N NaOH may be further added to suitably adjust the pH, e.g. to a final pH of about 5.5. The reaction may be allowed for a suitable duration, e.g. about 40 min at about 4° C.

Scheme 4 below shows a synthesis of DZ-1-Lysine-DOTA (Method 1):

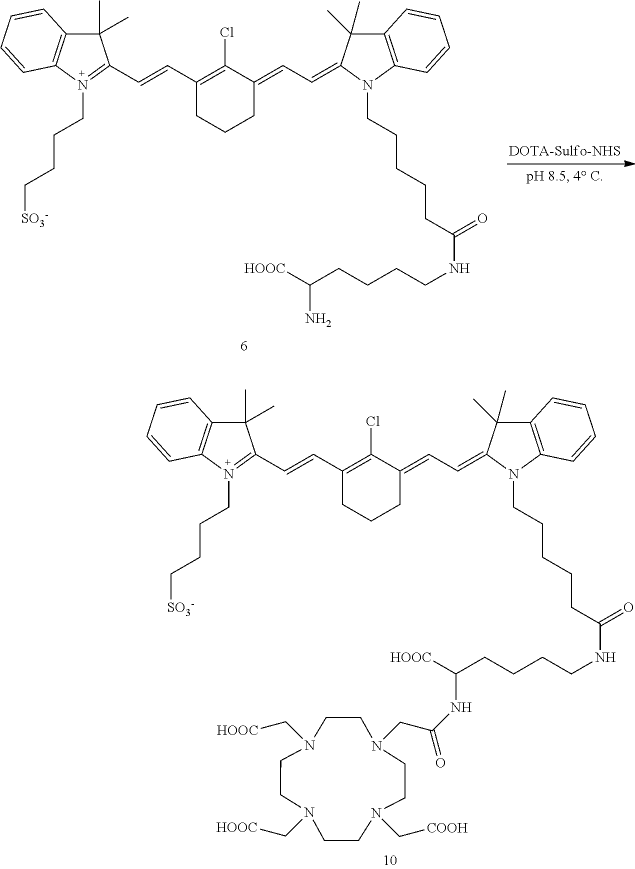

Synthesis of DZ-1-lysine-DOTA 10 (compare scheme 4 above): DZ-1-Lysine 6 (10 mg, 12 μmol) in 200 μL of 50% aqueous acetonitrile may be added to the DOTA-Sulfo-NHS 9 reaction mixture, and the pH may be adjusted to a suitable basic pH, e.g. to about 8.5, e.g. with 0.1 N NaOH. The reaction may be allowed to incubate for a suitable duration, e.g. overnight, at a sufficiently cool temperature, e.g. about 4° C. The DZ-1-DOTA conjugate may be purified by suitable separation methods, e.g. by HPLC, e.g. using an Apollo C18 semi-preparative column (e.g. 5 m, 250×10 mm). Two mobile phases may be used for HPLC: Solvent A (0.1% TFA in water) and Solvent B (0.1% TFA in 80% aqueous acetonitrile). The mobile phase gradient may be varied from 40% B to 100% B over a period of about 30 min at a flow rate of about 3 ml/min monitored at dual wavelengths 254 and 780 nm. The product may be collected and then lyophilized: 4 mg (28%). Mass spectrum (ESI) 1219.585 [M+H]⁺.

Scheme 5 below shows an alternative synthesis of DZ-1-Lysine-DOTA (method 2):

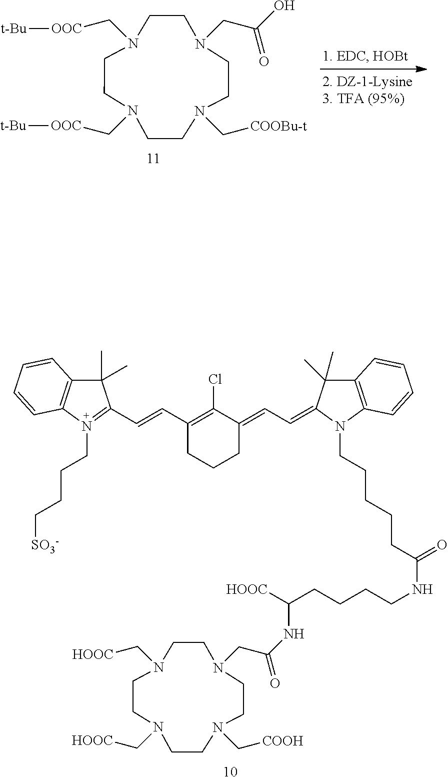

Alternatively, a synthesis of DZ-1-lysine-DOTA 10 may be performed as follows (method 2): The mixture of DOTA-tris (t-Bu ester) 11 (50 mg, 0.087 mmol)N-Ethyl-N′-(3-dimethylaminopropyl)carbodiimide hydrochloride (25 mg, 0.13 mmol) and 1-hydroxy-7-azabenzotriazole (15 mg, 0.11 mmol) may be dissolved in 3 ml DMF. The mixture may be stirred for 40 min, then DZ-1-Lysine 6 (73 mg, 0.087 mmol) may be added and stirred for a suitable time, e.g. about 5 additional hours at RT. The product may be precipitated, e.g. in cold diethyl ether (e.g. about 40 ml). Recovery may be by centrifugation for sufficient time at sufficient RPM, e.g. 5 min at 3500 rpm. The pellet which may be dissolved in a suitable solvent of suitable volume, e.g. in about 2 ml of TFA (95%), and stirred for sufficient time, e.g. about 3 hours. The crude product may be precipitated, e.g. in cold diethyl ether (e.g. about 40 ml) and purified, e.g. by C18-RP semi-preparative, to afford DZ-1-Lysine-DOTA 10 as a dark green solid 33 mg (31%).

### Example 1b—Labelling of DZ-1-Lys-DOTA Conjugate to Form DZ-1-Lys-DOTA-M (Here: Lu-177)

DZ-1-Lys-DOTA conjugates may be labelled, i.e. chelated, with radiometal metals (“M”) in any convenient way as will be apparent to a person of ordinary skill; for example, a radiometal metal M may be introduced in its ionic form, with the cationic radiometal (M e.g. selected from, without limitation: ¹⁷⁷Lu³⁺, ⁹⁰Y³⁺, ⁶⁸Gd³⁺) being paired with an anionic reagent such as a chloride (i.e. Mⁿ⁺Clⁿ) as shown in scheme 6 below, to form, e.g., without limitation: ¹⁷⁷LuCl₃, ⁹⁰YCl₃, ⁶⁸GaCl₃.

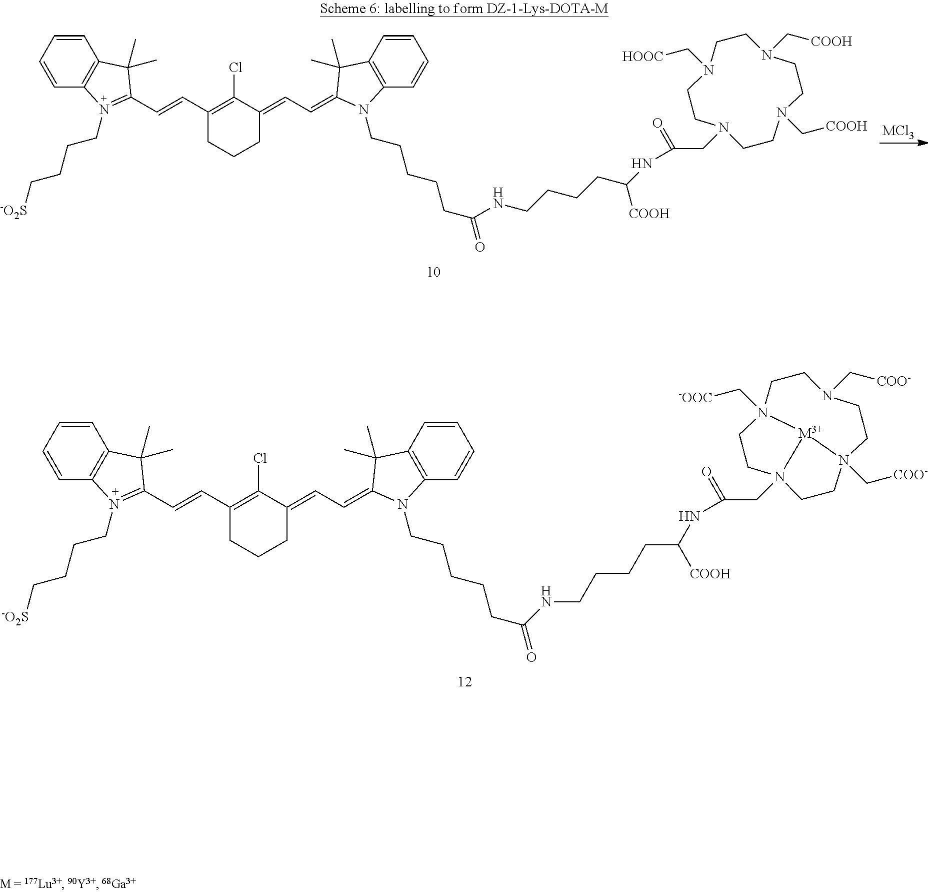

For radiolabeling, a suitably radioactive amount of radiometal may be added to the DZ1-Lys-DOTA conjugates, for example in a ratio of about 2 to about 200 MBq/μg (radiometal:conjugate), about 4 to about 100 MBq/μg or about 8 to about 50 MBq/μg e.g. about 20 MBq/μg. For example, 100 MBq (2.7 mCi) of ¹⁷⁷LuCl₃ to 5 μg of DZ-1-Lys-DOTA may be added to 0.1N ammonium acetate (pH 5.5) buffer, and the mixture may be incubated at a suitable temperature for a sufficient time to achieve complete labelling, for example at about 20° C. to about 60° C., or about 30° C. to about 50° C., e.g. at about 40° C. for about 10 to about 60 minutes or about 20 to about 40 minutes, e.g. about 30 minutes.

For example, radiolabeling may be accomplished by addition of 100 MBq (2.7 mCi) of ¹⁷⁷LuCl₃ to 5 μg of DZ-1-Lysine-DOTA in 0.1N ammonium acetate (pH 5.5) buffer, and incubating the mixture at about 40° C. for about 30 min. The DZ-1-Lysine-DOTA-¹⁷⁷LuCl₃ complex may be purified by Reversed-phase HPLC with an Apollo C18 RP column (5 p, 250×10 mm). The column eluate may be monitored by ultraviolet absorbance at 254 nm and with a NaI crystal detector. The mobile phase may change from 40% solvent A (0.1% trifluoroacetic acid in 80% water) and 60% solvent B (0.1% trifluoroacetic acid in 80% aqueous acetonitrile) to 100% solvent B at 30 min at a flow rate of 3 ml/min. The pure fraction of DZ-1-Lys-DOTA-M³+Cl³ complex from the HPLC may be concentrated by gently blowing a positive flow of nitrogen for drying. The residue left in test tube upon concentration may be reconstituted in a suitable buffer depending on further testing or administration, e.g. 1×PBS buffer (1.0 ml).

DZ-1-Lysine-DOTA complexes of ⁹⁰Y³⁺ and ⁶⁸Ga³⁺ may be formed and labeled using a similar protocol, e.g. using ⁹⁰YCl₃ or ⁶⁸GaCl₃, as will be apparent to a person of ordinary skill.

### Example 2: Targeted Radiotherapy, Tumor Weight Reduction and Retained Body Weight in Mice

To assess the therapeutic effects, efficacy and toxicity of 177Lu-DZ-1-Lysine-DOTA, nude mice may be inoculated subcutaneously with human brain cancer cells (e.g. neuroblastoma, astrocytoma, or glioblastoma cells, publicly available e.g. from ATCC). Subsequently, nude mice with a subcutaneous tumor size of e.g. about ˜100 mm³ may be randomly assigned to several groups (e.g. n=5 mice per group). For the therapeutic group, tumor-bearing mice will be administered intravenously (e.g. via the tail vein) of e.g. about 0.2 ml of 177Lu-DZ-1-Lysine-DOTA (5.55 GBq/kg). The treatment may be given in regular intervals, e.g. once a day or week for several days or weeks, e.g. once a week for 4 weeks. With the same treatment scheme, a control group may be injected via tail vein with vehicle solvent (e.g. saline of the same volume, e.g. 0.2 ml). After the treatment, mice may be kept for another period, e.g. 12 weeks. Endpoint of the study will be differential tumor growth between treatment and control group. Body weight may be monitored throughout.

Alternatively the therapeutic effects, efficacy and toxicity of 177Lu-DZ-1-Lysine-DOTA may be determined essentially using nude mice as described above except that inoculating with human brain cancer cells is performed intracranially, and the endpoint of the study will be differential animal death between treatment and control group. Body weight may be monitored throughout.

Tumor specific targeting of the 177Lu-DZ-1-Lysine-DOTA may be determined with one or more methods, e.g. 1) near infrared fluorescence tumor imaging to detect accumulation of the DZ-1 moiety in the tumor, and 2) SPECT/CT nuclear imaging of the 177Lu in the same tumor. Tumor dimensions may be measured in regular intervals, e.g. daily or once or twice weekly, with a suitable method such as with calipers, e.g. digital calipers, and the tumor volume may be calculated using the formula: volume=½ (length×width×width). To monitor potential toxicity, body weight may be measured. Mice may be euthanized when the tumor size exceeded the volume of 1,500 mm³ or the body weight lost >20% of original weight. Complexes as described herein may advantageously show a decreased tumor weight, indicative of efficacy, and retained body weight and indicative of a lack of toxicity.

Alternatively to 177Lu-DZ-1-Lysine-DOTA, 90Y-DZ-1-Lysine-DOTA may be used in the protocols as described above.

### Example 3: PET/MicroPET Imaging

PET (or microPET) may be performed before, after, or during therapy with 177Lu- or 90Y-complexes as described herein, to provide image guided therapy. Without wishing to be bound by theory, it is believed that combination with 177Lu, 90Y, or both, provides a well matched true theranostic pair that allows the therapeutic radiometal to destroy the tumor while guided by the imaging radiometal, which may be due to their pharmacokinetics, stability and efficacy, including similar binding affinities, tumor uptake and organ clearance.

MicroPET imaging is PET performed on small animals, as will be apparent to a person of ordinary skill. The DZ-1-Lysine-DOTA labeled with Ga-68 may be administered e.g. intravenously to individual mice, e.g. in a set of five by injecting e.g. about 300-500 μCi of the complex. The Transaxial microPET images may be collected at suitable time intervals, e.g. at 1, 2 and 3 h post probe injection (pi) time points. The standardized uptake value (SUV) analyses may be performed on cancer xenografts and skeletal muscles of individual mice as defined e.g. by CT scans and the tumor-to-muscle ratio may be calculated for each group at these time points. Blood clearance and organ distribution: after anesthesia (e.g. by isoflurane 2-3%), a group of e.g. 5 mice may be injected with radiolabeled 68Ga-DZ-1-Lysine-DOTA complexes (˜5 μCi), e.g. via the tail vein. Retro-orbital blood samples (25 μl) may be collected at various time points e.g. at 5, 15, 30, 60 and 180 min after injection, and radioactivity of all samples may be counted in a gamma counter (e.g. 1480 Wizard, Perkin-Elmer) and normalized to plot against injection time and followed by a non-linear regression analysis to obtain the half-life time in blood (e.g. by Prism™ software, e.g. version Prism 9, publicly available from GraphPad™, San Diego, CA). Mice may be sacrificed immediately after the last blood sampling. Tumors and organs (such as heart, liver, lung, kidney, small intestine, stomach, bone, muscle, spleen and skin) may be harvested and counted for radioactivity in a gamma counter. Organ distribution data in duplicate may be obtained for DZ-1-Lysine-DOTA complexes in mice at a plurality of time points, e.g. three time points, e.g. at 1 h, 2 h and 3 h. Image registration and analysis may be performed as follows: PET/CT images may be processed following a standard protocol as will be apparent to a person of ordinary skill, e.g. using standard software as per the manufacturer's guides, e.g. using the ASIPro™ software, Siemens Healthineers™, Erlangen, Germany. Pixel-wise standardized uptake values (SUVs) of PET may be calculated as product of the pixel-wise activity divided by the injected dose and body weight. The tumor target may be delineated as 40% of maximum of SUV, anatomically overlaying with a CT image through image registration through the software used. Complexes as described herein may advantageously show a specific distribution characterized by high uptake in the tumors. The distribution ratio between tumor and skeletal muscle may be e.g. around 8:1 after 24 h and may be e.g. above 20:1 after 48 h of 68Ga-DZ-1-Lysine-DOTA administration.

### Example 4: Determination of Serum Stability of the Lu-177, Y-90 and Ga-68 Complexes

The serum stability of the complexes described herein may be determined as follows. A suitable amount, e.g. 50 microcuries of a complex as described herein, e.g. of a DZ-1-Lysine-DOTA radio-metal complex with Lu-177, Y-90 or Ga-68, may be added into a suitable volume, e.g. 100 μl, of fetal bovine serum (Invitrogen, Grand Island, NY). After incubation at 37° C. for suitable time intervals, e.g. 1, 3, and 6 h, aliquots of the mixture may be removed and filtered, e.g. through a 0.2 μM microspin filter. The resulting filtrates may be analyzed by a suitable separation and detection method, e.g. by reverse-phase HPLC with a Bioscan Flow Count Radio-HPLC detector. The original peak represents an undegraded stable complex (here a DZ-1-Lysine-DOTA complex with Lu-177, Y-90 or Ga-68, compare e.g. compound 12 shown herein-above). Detection of any newly formed 7-peaks in addition to the original peak identifies degraded products and thus a lack of stability of the complex in the serum. Analysis of a corresponding complex with Cu-64 indicates a lack of stability for the Cu-64 complex, in contrast to the Ga-68, Lu-177, or Y-90 complexes, in particular DZ-1-Lysine-DOTA-Lu-177, DZ-1-Lysine-DOTA-Y-90, and DZ-1-Lysine-DOTA-Ga-68. Lu-177, Y-90 and Ga-68 complexes may thus provide superior serum stability.

### Example 5: Image-Guided Therapy of Prostate Cancer

Male nude mice of 4-6 weeks of age may be inoculated subcutaneously with human prostate cancer cells, e.g. C4-2B (also known as ATCC® CRL-3315™ and publicly available from American Type Culture Collection (ATCC), Manassas, Virginia) or ARCaP_{M}, publicly available from Novicure™, Birmingham, AL (ARCaPM cells, Catalog Number: 3422, are human prostate cancer cells established from a parental mixed ARCaP cell population with high propensity for bone metastasis in mice. Histopathology of the tumors in bone is mainly of osteoblastic lesions that recapitulate human prostate cancer bone metastasis. ARCaPM cells (spindle-shape mesenchymal morphology) were derived by single cell cloning of the parental ARCaP cells. ARCaPM are highly aggressive prostate cancer metastatic cells. In one study, the incidence of bone metastasis for ARCaPM cells, after intracardiac injection, was determined to be 100% (9/9) with a respective latent period of 71- and 61-days. ARCaPM cells can grow in culture using MCaP culture Medium available from Novicure™. ARCaPM cells may be used to study prostate cancer bone metastasis and the role of EMT in cancer metastasis. MCaP-medium was prepared using Dulbeccos modified eagle and F12K medium and contains essential and non-essential amino acids, vitamins, organic and inorganic compounds, hormones, growth factors and trace minerals and supplemented with several factors that are critical for the optimal growth of ARCaP cells in vitro. The medium is serum-free and should be supplemented with 5% heat-inactivated Fetal Bovine Serum. It is bicarbonate buffered and has a pH of 7.4 when equilibrated in an incubator with an atmosphere of 5% CO₂/95% air).

The mice may be kept for a suitably long duration, e.g. 2 weeks, for tumor formation to about 100 mm³ in volume. The probe solution for injection will be prepared in sterile phosphate buffered saline (PBS) (radioactive doses of probe DZ-1-Lysine-DOTA ⁶⁸Ga complex). Mice may be administered intravenously (e.g. via tail vein) with the imaging probe in a suitable volume (e.g. a volume of 100-150 l), followed by procedures of blood collection, probe biodistribution, and fluorescence and PET imaging under inhalation anesthesia (2% isoflurane in oxygen), as will be apparent to a person of ordinary skill.

As a first optional step, Ga-68-DZ-1-Lysine-DOTA complexes as described herein may be administered e.g. as described herein to detect a primary tumor, and/or may be used to detect any residual metastases or secondary tumors thereof, e.g. after removal of a primary tumor, e.g. by surgery.

Therapy may be performed on a first and/or one or more secondary tumor, or metastases of a first or one or more secondary tumor using Lu-177 or Y-90 complexes as described herein, optionally with parallel detection of residual metastases/tumors by Ga-68, optionally followed by a first tumor therapy, or in case a first tumor therapy has been performed, a second or further tumor therapy, applying Lu-177 or Y-90 complexes as described herein for image-guided radiotherapy. Complexes described herein may be particularly advantageous for imaging, therapy, and/or image-guided therapy of prostate cancer, including e.g. an improved serum stability, reduced toxicity, and significantly reduced metastatic bone tumor formation.

### Example 6: Image-Guided Therapy of Brain Tumors Including Glioblastoma

To determine tumor suppression efficacy and/or animal survival and/or body weight etc., experiments may be essentially performed as described herein-above under example 2. For example, to assess tumor suppression efficacy, nude mice may be inoculated subcutaneously with human brain cancer cells (e.g. neuroblastoma, astrocytoma, or glioblastoma cells, publicly available from ATCC). Subsequently, nude mice with subcutaneous tumor size of ˜100 mm³ may be randomly assigned to several groups (e.g. n=5 mice per group). To assess the efficacy on animal survival, the therapeutic effects, efficacy and toxicity may be determined by inoculating nude mice intracranially with human brain cancer cells (e.g. neuroblastoma, astrocytoma, or glioblastoma cells publicly available from ATCC). One week after inoculation, the subject mice will be randomly assigned to several groups (e.g. n=5 mice per group).

The probe solution for injection may be prepared in sterile PBS buffer. Mice may be administered intravenously (via tail vein) with imaging probes (PBS volume e.g. 100-150 l) for blood collection, probe biodistribution, and fluorescence and PET imaging by intravenous injection e.g. via the mouse tail vein, e.g. using a syringe (e.g. ½ cc U-100 Insulin Syringe) under inhalation anesthesia (2% isoflurane in oxygen). Radioactive doses, also known as probe, may be the DZ-1-Lysine-DOTA ⁶⁸Ga complex, formed as will be apparent to a person of ordinary skill and as generally described herein-above.

One or more treatment administrations, optionally preceded or followed by one or more imaging step administration may be generally performed as described in example 5 herein-above, with the following adjustments: The probe solution for injection may be prepared in sterile PBS buffer. Mice may be administered with imaging probes (PBS volume 100-150 l) for blood collection, probe biodistribution, and fluorescence and PET imaging e.g. by intravenous injection e.g. via mouse tail vein e.g. using a syringe (e.g. ½ cc U-100 Insulin Syringe) under anesthesia (2% isoflurane in oxygen). Radioactive doses of probe may be a DZ-1-Lysine-DOTA ⁶⁸Ga complex.

In a first step, a brain tumor that is inoculated in the subcutaneous space may be surgically removed, optionally after detection using Ga-68 complexes as described herein. In a second step, residual disease may be determined post-surgery and/or at intervals after surgery to detect resurgence using Ga-68 complexes as described herein. Upon detection of residual or resurgent metastases or tumors, treatment with Lu-177 or Y-90 complexes may be initiated.

In animals bearing intracranial brain tumor, the first step may be detection of the tumor formation with Ga-68 complexes as described herein which may determine that the complex crosses the blood-brain-barrier. The second step may be treating the brain tumor with Lu-177 or Y-90 complexes to prevent or slow tumor growth, prevent or postpone animal death, and/or cause tumor shrinkage.

Complexes described herein may be particularly advantageous for imaging, therapy, and/or image-guided therapy of glioblastoma, including e.g. an improved serum stability, while retaining the ability to cross the blood-brain-barrier to improve animal survival.

### Example 7: Testing Complexes for Specificity and Tumor Tissue Penetration by Imaging Methods Such as PET

Prior to administration to subjects in need thereof, the specificity and tumors tissue penetration of the dye-chelator complexes described herein may be determined by any suitable conventional method, including e.g. PET/CT imaging, e.g. using a suitable animal model, as will be apparent to a person of ordinary skill. For example, a mammalian model such as a mouse model, in particular, a xenograft mammalian model having the cancer of interest, e.g. a mouse prostate cancer xenograft model, may be used for testing of the conjugates by imaging, in particular, PET imaging. Other mammalian models may be used, as will be apparent to a person of ordinary skill (e.g. rat, rabbit, dog, monkey, etc.).

A radiometal complex as herein described may be administered as will be apparent to a person of ordinary skill. For example, without limitation, administration may be performed intraperitoneally to individual mammals (here: mice), e.g. in a set of five, by injecting 300-500 μCi of the complex. Transaxial microPET images may be collected at regular intervals in time, e.g. at 12, 24 and 48 h post probe, i.e. complex, injection (pi) time points. Standardized uptake values (SUV) analyses may be performed as will be apparent to a person of ordinary skill, here on cancer xenografts and muscles of individual mice as defined by CT scans, and a tumor-to-muscle ratio may be calculated for each group at these time points, as will be apparent to a person of ordinary skill.

Complexes may provide one or more advantages including an increased specificity as shown by PET imaging, e.g. the Standardized uptake values (SUV) in the tumor may be improved, tumor retention and/or tumor penetration may be increased e.g. as shown by PET/CT. For example, the tumor-to-muscle ratio after about 24 h of administration may be about 8:1 and may be significantly increased by about 20% or more (10:1), about 30% or more, about about 40% or more, about 50% or more, about 60% or more, about 70% or more, about 80% or more, about 90% or more, about 100% or more, about 120% or more (20:1), after about 36 to about 48 h of administration, or longer, as the tumor tissue retains the conjugate for an extended time.

### Example 8: Image Registration and Analysis of PET/CT Images, Determination of SUV

PET/CT images may be processed following a standard protocol, as will be apparent to a person of ordinary skill. Pixel-wise standardized uptake values (SUVs) of PET may be calculated as a product of the pixel-wise activity divided by the injected dose and body weight of the subject. The tumor target may be delineated as 40% of maximum of SUV by anatomically overlaying the delineated tumor with a corresponding CT image through image registration.

### Example 9—Determination of Blood Clearance and Organ Distribution of a Complex

After anesthesia (e.g. by administration of isoflurane 2-3%), a suitably sized group of mammals, here: 5 mice, may be injected with one or more complex, e.g. radiolabeled DZ-1-Lys-DOTA (DZ-1-Lys-DOTA-¹⁷⁷LuCl₃, DZ-1-Lys-DOTA-YCl₃, vDOTA-GaCl₃), for example each at a dose of about 5 μCi, via a suitable administration method, e.g. via a suitable vein, here e.g. the tail vein. Blood samples, e.g. retro-orbital blood samples of sufficient size (e.g. about 25 μl) may be collected at suitable time points in regular intervals, e.g. at 5, 15, 30, 60, 180, 360 min and 24 h after injection of the complex. Radioactivity of all samples may be counted as apparent to a person of ordinary skill, e.g. in a gamma counter (e.g. 1480 Wizard™, Perkin-Elmer™ Waltham, MA), and results may be normalized to plot against injection time, followed by non-linear regression analysis to obtain the half-life time in blood, e.g. in hours. Test animals, here: mice, may be sacrificed immediately after the last blood sampling. Tumors and organs (such as heart, liver, lung, kidney, small intestine, stomach, bone, muscle, spleen and skin) may be separately harvested and organ-specific radioactivity and/or half-live and/or organ residence time may be determined essentially as described above. Determination may show that DZ-1-Lys-¹⁷⁷Lu-177, DZ-1-Lys-DOTA-Y-90, DZ-1-Lys-DOTA-Ga-68 have a favorable blood and organ clearance. A comparison of these complexes to other complexes, in particular e.g. to DZ-1-Lys-DOTA-Cu-64 complexes, may show superior clearance.

### Example 10: ⁶⁸Ga Radiolabeling

DZ-1-Lys-DOTA was labeled with ⁶⁸Ga by the addition of ⁶⁸GaCl₃ fresh eluent (0.1 M, 1-3 mCi) to a solution of DZ-1-Lys-DOTA (50 μg) in sodium acetate solution (0.1N, pH 3-4). The reaction mixture was incubated at 95° C. for 15 min. After semi-preparative HPLC purification (5 m, 250×10 mm), the radiochemical purity of DZ-1-Lys-DOTA-⁶⁸Ga was determined by Radio-HPLC (93.25±0.98%) which showed a single major peak identifying DZ-1-Lys-DOTA-6Ga.

### Example 11: Serum Stability of DZ-1-Lys-DOTA-⁶⁸Ga

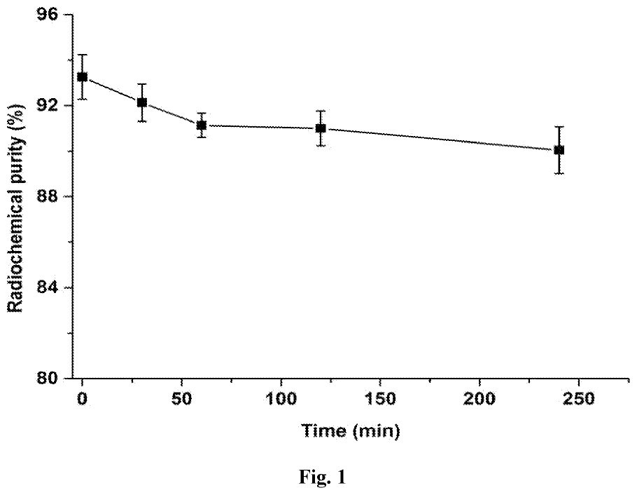

DZ-1-Lys-DOTA-⁶⁸Ga was incubated with Fetal Bovine Serum (FBS) at 37° C., and the stability of DZ-1-Lys-DOTA-⁶⁸Ga was analyzed by radio-TLC with 0.1M citric acid as eluent. The radiochemical purity of the sample was monitored during four half-lives, as shown in FIG. 1.

The % purity is a marker for stability, with a higher purity indicating a higher stability. A retention of above 80% purity within 4 hours demonstrates excellent stability.

As shown in FIG. 1, the initial stability as measured by radiochemical purity was about 93%, and after four half-lives remained stable at about 90% at about 240 minutes or 4 hours; the detected amounts over time are shown in the table below.

TABLE 1
Serum Stability of DZ-1-Lys-DOTA-68Ga

| Time (min) | 0 | 30 | 60 | 120 | 240 |
|---|---|---|---|---|---|
| Radiochemical | 93.25 ± | 92.13 ± | 91.13 ± | 91.00 ± | 90.04 ± |
| purity (%) | 0.98 | 0.82 | 0.53 | 0.76 | 1.03 |

### Example 12: Blood Clearance of DZ-1-Lys-DOTA-⁶⁸Ga

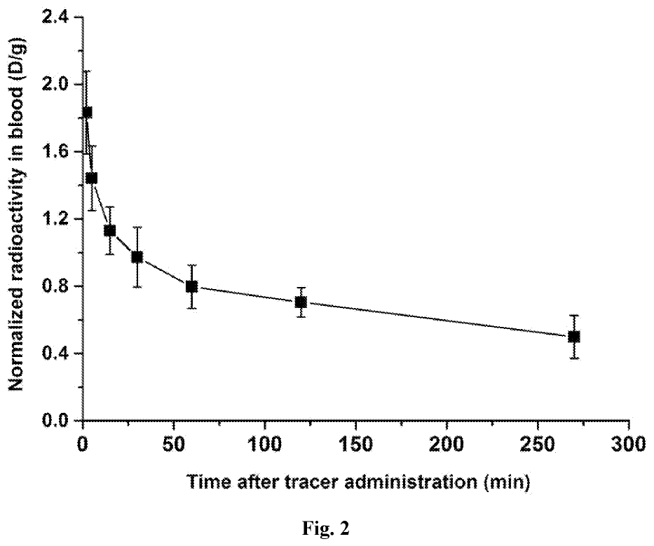

DZ-1-Lys-DOTA-⁶⁸Ga (50 μCi) probe was injected into the tail vein of Balb/C mice (n=6, each about 20 g of body weight). Approximately 10 μL of blood from the contralateral tail vein was collected in capillary tubes at 2 min, 5 min, 15 min, 30 min, 1 h, 2 h and 4.5 h post probe injection. The capillaries were placed in pre-weighed tubes to measure the weight and radioactivity of the blood withdrawn. Radioactivity from each sample was measured by a gamma counter with radioactivity decay corrected to the time of probe injection, normalized for injected dose and blood weight, and expressed as percentage injected dose per gram blood (FIG. 2). Each animal was i.v. injected 50 μCi of the probe.

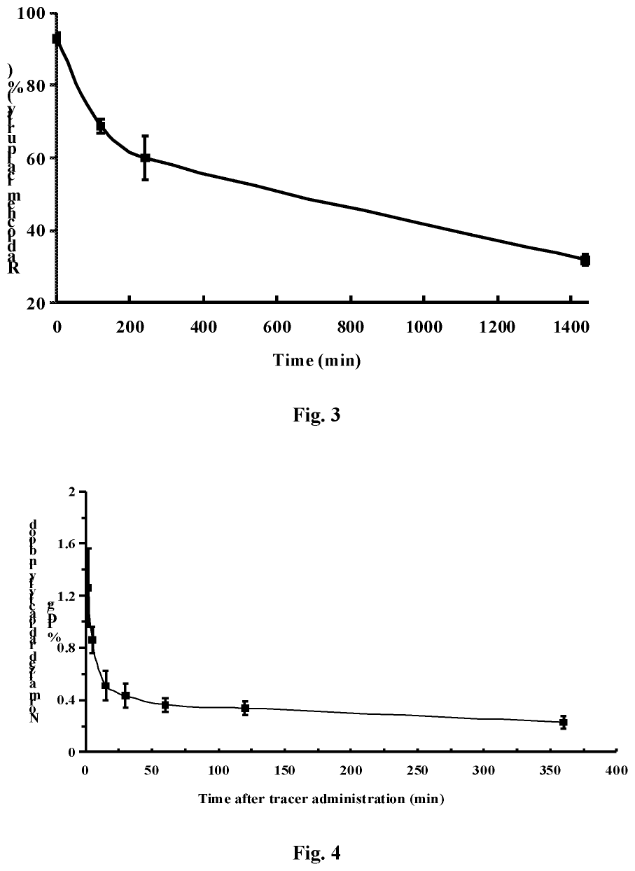

As shown in FIG. 3, the blood clearance measured as normalized radioactivity in blood (D/g) formed a decreasing curve that decreased from about 1.8 to about 0.5 within 270 minutes, with an initial rapid decrease to about 0.8 during about 60 minutes. This shows that DZ-1-Lys-DOTA-⁶⁸Ga is stable at least up to 270 minutes and longer, also see table 2 below. The elimination half-life for blood clearance (T1/2) was calculated to be 30 min.

TABLE 2
Blood Clearance of DZ-1-Lys-DOTA-68Ga

| Time (min) | 2 | 5 | 15 | 30 | 60 | 120 | 270 |
|---|---|---|---|---|---|---|---|
| Normalized | 1.83 ± | 1.44 ± | 1.13 ± | 0.97 ± | 0.80 ± | 0.70 ± | 0.50 ± |
| radioactivity | 0.25 | 0.19 | 0.14 | 0.18 | 0.13 | 0.09 | 0.13 |
| in blood | | | | | | | |
| (% ID/g) | | | | | | | |

### Example 13: 17Lu Radiolabeling

DZ-1-Lys-DOTA was labeled with ¹⁷⁷Lu by the addition of ¹⁷⁷LuCl₃ (1-3 mCi, 0.1M, 200 μL) to a solution of DZ-1-Lys-DOTA (50 μg) in sodium acetate (0.1M, pH 4-5). The reaction mixture was incubated at 95° C. for 15 min. The radiochemical purity of the label was detected by radio-Thin Layer Chromatography (TLC) with 0.1M citric acid as eluent (92.06±1.17%). The probe was sufficiently pure and thus stable at about 92%.

### Example 14: Serum Stability of DZ-1-Lys-DOTA-¹⁷⁷Lu

The DZ-1-Lys-DOTA-¹⁷⁷Lu was incubated with Fetal Bovine Serum (FBS) at 37° C., and the probe was analyzed by radio-TLC with 0.1M citric acid as eluent (see FIG. 3). Stability is determined by the percentage of the labeled compound (radiochemical purity).

As shown in FIG. 3 and the table below, DZ-1-Lys-DOTA-¹⁷⁷Lu is stable as apparent from the purity, i.e. stability which decreased over time from more than 90% at the start of the incubation to about 60% after 240 minutes, about 50% after 700 minutes, and about 30% at 1440 minutes. This shows the DZ-1-Lys-DOTA-¹⁷⁷Lu has sufficient stability for clinical use.

TABLE 3
Serum Stability of DZ-1-Lys-DOTA-177Lu

| Time (min) | 0 | 120 | 240 | 1440 |
|---|---|---|---|---|
| Radiochemical | 92.87 ± 1.86 | 68.75 ± 1.96 | 60.04 ± 5.93 | 31.83 ± 1.56 |
| purity (%) | | | | |

### Example 15: Blood Clearance of DZ-1-Lys-DOTA-¹⁷⁷Lu

DZ-1-Lys-DOTA-¹⁷⁷Lu (50 μCi) was injected into the tail vein of Balb/C mice (n=6, about 20 g). Approximately 10 μL of blood from the contralateral tail vein was collected in capillary tubes at 2 min, 5 min, 15 min, 30 min, 1 h, 2 h and 6 h post probe injection. The capillaries were placed in pre-weighed tubes to measure the weight and radioactivity of the blood withdrawn. Radioactivity from each sample was measured by a gamma counter with the radioactivity decay corrected to the time of probe injection, normalized for injected dose and blood weight, and expressed as percentage injected dose per gram blood.

A graph of the results is shown in FIG. 4, results are shown in the table below. The elimination half-life for blood clearance (T1/2) was calculated to be 15 min. A rapid decrease and thus rapid blood clearance from about 1.25 to 0.5% D/g normalized radioactivity in blood in less than about 20 minutes, and below 0.4 in less than about an hour, shows a fast removal of the complex from circulation; this is believed to be at least partially caused by successful tumor-specific uptake of the complex in the Balb/C mice which are prone to the development of cancer and tumors.

TABLE 4
Blood Clearance of DZ-1-Lys-DOTA-177Lu

| Time (min) | 2 | 5 | 15 | 30 | 60 | 120 | 360 |
|---|---|---|---|---|---|---|---|
| Normalized | 1.26 ± | 0.86 ± | 0.51 ± | 0.43 ± | 0.36 ± | 0.34 ± | 0.23 ± |
| radioactivity in | 0.30 | 0.10 | 0.11 | 0.10 | 0.05 | 0.05 | 0.05 |
| blood (% ID/g) | | | | | | | |

### Example 16: ⁹⁰Y Radiolabeling

DZ-1-Lys-DOTA was labeled with ⁹⁰Y by the addition of ⁹⁰YCl₃ (1˜3 mCi, 0.1M, 200 μL) to a solution of DZ-1-Lys-DOTA (50 μg) in sodium acetate (0.1M, pH 4-5). The reaction mixture was incubated at 95° C. for 30 min. The radiochemical purity of the label was detected by radio-TLC with 0.1M citric acid as eluent at 86.69±1.17% as a single main peak, with a minor secondary peak present which can be easily purified by HPLC.

### Example 16: Serum Stability of DZ-1-Lys-DOTA-⁹⁰Y

The DZ-1-Lys-DOTA-⁹⁰Y was incubated with Fetal Bovine Serum (FBS) (V:V=2:1) at 37° C. and the probe was analyzed by radio-TLC with 0.1M citric acid as eluent.

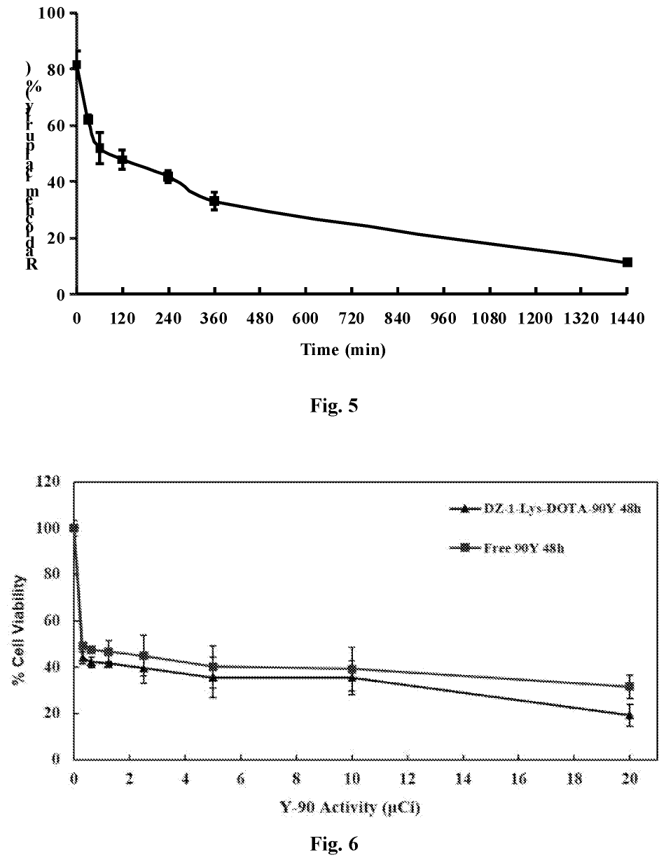

As shown in the graph of FIG. 5 and the table below DZ-1-Lys-DOTA-90Y is stable at about 50% purity/stability for at least about an hour, at above 40% for about 4 hours, retains at least 30% stability and/or purity for at least about 6 hours, and at least about 10% for at least 24 hours.

TABLE 5
Serum Stability of DZ-1-Lys-DOTA-90Y

| Time (min) | 0 | 30 | 60 | 120 | 240 | 360 | 1440 |
|---|---|---|---|---|---|---|---|
| Radiochemical | 81.50 ± | 62.04 ± | 51.86 ± | 47.80 ± | 41.90 ± | 33.01 ± | 11.47 ± |
| purity (%) | 4.71 | 1.45 | 5.45 | 3.34 | 2.06 | 3.10 | 1.40 |

### Example 17: Cytotoxicity Study

The MDA-MB-231 breast cancer cells were pre-seeded into 96-well plates (10⁴ cells/well), and placed in a 37° C., 5% CO₂ incubator until the cells were fully attached. Serially diluted DZ-1-Lys-DOTA-⁹⁰Y (100 μL) was added to each well (7 concentrations diluted from 20 μCi in half), and 3 replicate wells for each concentration point. After 24 and 48 hours of treatment, the supernatant was discarded, and 10 μL of CCK-8 solution and 100 μL of medium were added to each well. The culture was continued for 1 h in the incubator, and the absorbance of each well at 450 nm was measured with a microplate reader.

As shown in FIG. 6, and the table below, the results show that DZ-1-Lys-DOTA-⁹⁰Y kills cancer cells more effectively compared to the free ⁹⁰Y. A 20 μCi of the complex caused 80% of the breast cancer cell to die within 48 hours. This is believe to be a due to a combination of its stability and higher uptake of the complex by cancer cells compared to free 90Y.

TABLE 6
Cytotoxicity of DZ-1-Lys-DOTA-⁹⁰Y

| Y-90 Activity | 0 | 0.3125 | 0.625 | 1.25 | 2.5 | 5 | 10 | 20 |
|---|---|---|---|---|---|---|---|---|
| DZ-1-Lys- | 100.00 ± | 43.92 ± | 42.08 ± | 41.50 ± | 39.63 ± | 35.57 ± | 35.46 ± | 19.21 ± |
| DOTA-90Y | 3.36 | 2.59 | 2.13 | 0.80 | 6.56 | 8.82 | 7.38 | 4.61 |
| (48 h) | | | | | | | | |
| Free 90Y | 100.00 ± | 49.23 ± | 47.59 ± | 46.71 ± | 44.94 ± | 40.15 ± | 39.27 ± | 31.56 ± |
| (48 h) | 3.36 | 0.92 | 1.05 | 4.63 | 8.66 | 9.03 | 9.43 | 5.03 |

Many suitable methods to make or use the complexes described herein are known in the art. According to an embodiment of the present invention, the complexes may be used for cancer therapy as described herein-above. Many different options of administration and treatments are available and can be selected and applied depending on the individual subject and determined treatment protocol, as will be apparent to a person of ordinary skill. Features of one embodiment may be employed with other embodiments as the skilled artisan would recognize, even if not explicitly stated herein. Descriptions of well-known components and processing techniques may be omitted so as to not unnecessarily obscure the embodiments.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the descriptions are to be regarded as illustrative in nature rather than restrictive.

1. A DZ-1-Lys-DOTA conjugate radiometal complex, wherein the conjugate comprises a heptamethine carbocyanine dye (HMCD) moiety conjugated with a DOTA moiety via a lysine linker and as shown in FI below: 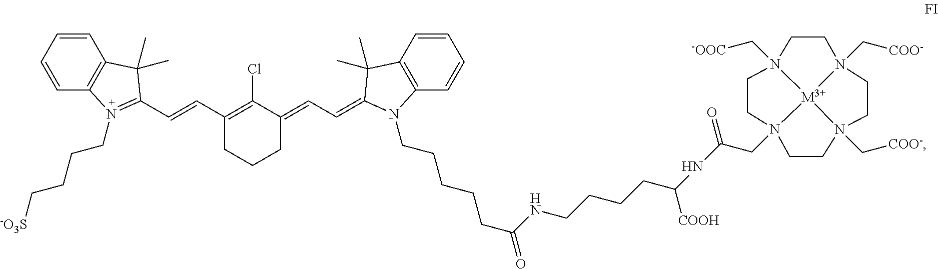
wherein the DOTA moiety is complexed with a radiometal M,
and wherein M is selected from one or more radiometal from the group consisting of: Lutetium-177 (Lu-177), Yttrium-90 (Y-90), and Gallium-68 (Ga-68), or a combination of one or more of such complexes, the combination comprising a combination of Gallium-68 with Lutetium-177, and a combination of Gallium-68 with Yttrium-90.

2. The complex of claim 1 provided with one or more pharmaceutically acceptable excipient as one or more pharmaceutical formulation.

3. The complex of claim 2, wherein one or more pharmaceutical formulations are adapted for coordinated administration of a first and a second complex to provide an image-guided therapy of cancer, wherein the radiometal of the first complex is Gallium-68, and the radiometal of the second complex is selected from the group consisting of Lutetium-177 and Yttrium-90.

4. The complex of claim 3, provided as a kit with one or more reagents for reconstitution of the first and the second complex in an administrable form.

5. A kit for forming one or more complex from a DZ-1-Lys-DOTA conjugate of formula FII as shown below, or combinations thereof: 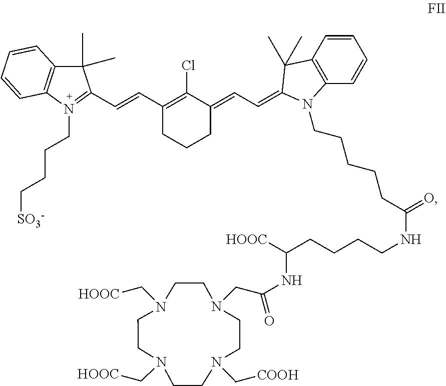
wherein the kit is provided with instructions for mixing and complexing the one or more conjugate in a suitable amount with the one or more radiometal selected from the group consisting of Lu-177, Y-90, and Ga-68 in a suitable amount, optionally with one or more reagent, buffer or excipient, and optionally treating the resulting solution containing the formed complex to provide it in an administrable form.

6. The kit of claim 5 for forming a plurality of complexes for coordinated administration of a first and a second complex, wherein the first complex is Ga-68, and the second complex is selected from the group consisting of Lu-177 and Y-90.

7. A method for image-guided therapy of cancer wherein a first and a second DZ-1-Lys-DOTA conjugate radiometal complex of formula I are administered in a coordinated administration schedule to a subject suffering from cancer to provide an image-guided therapy of cancer;
wherein the radiometal of the first complex is Gallium-68,
wherein the radiometal of the second complex is selected from the group consisting of Lutetium-177 and Yttrium-90;
and wherein the first and the second complex has the structure shown in FI below:

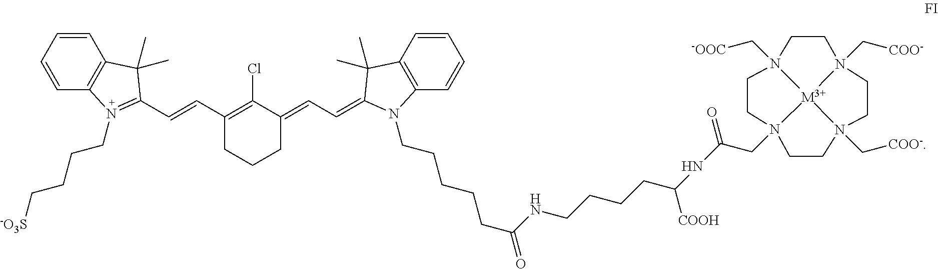

8. The method of claim 5, wherein imaging is performed by Positron Emission Tomography (PET) and optionally by PET and Computer Tomography (CT).

9. The invention substantially as described herein.
